(19) United States
(12) Patent Application Publication
(10) Pub. No.: US 2026/0283555 A1
(43) Pub. Date: Sep. 24, 2026

(54) CARDIAC DEVICE PLACEMENT AND VERIFICATION AID

(71) Applicants: ZOLL Medical Corporation, Chelmsford, MA (US); ZOLL Medical Israel Ltd., Kfar-Saba (IL)

(72) Inventors: Rafael Ravid, Pittsburgh, PA (US); Stephan D. Morgan, North Huntingdon, PA (US)

(73) Assignees: ZOLL Medical Corporation, Chelmsford, MA (US); ZOLL Medical Israel Ltd., Kfar-Saba (IL)

(21) Appl. No.: 19/567,519

(22) Filed: Mar. 16, 2026

Related U.S. Application Data
(60) Provisional application No. 63/773,356, filed on Mar. 17, 2025.

(51) Int. Cl. A61B 5/00

(52) U.S. Cl. CPC A61B 5/684; A61B 5/0002; A61B 5/6833; A61B 5/746

(57) ABSTRACT

A radio frequency (RF) based physiological monitoring device may include a patch configured to be adhesively coupled to skin of a patient, and an RF sensor configured to be mechanically coupled to the patch. The RF sensor includes at least one radio frequency (RF) antenna and associated transmitter/receiver circuitry configured to transmit RF waves into and receive reflected RF waves from a body of the patient. The device may include at least one processor in electrical communication with the at least one radio frequency (RF) antenna and associated transmitter/receiver circuitry and be configured to provide an alert that a re-placement of the patch at a proposed anatomical location is acceptable or unacceptable, apply a correction for RF-metrics determined by a re-placed device, and/or send a signal to recalibrate the re-placed RF-based physiological monitoring device.

### CROSS-REFERENCE TO RELATED APPLICATION

This nonprovisional application claims priority to U.S. Provisional Patent Application Ser. No. 63/773,356, filed on Mar. 17, 2025, titled “CARDIAC DEVICE PLACEMENT AND VERIFICATION AID,” the entirety of which is hereby incorporated by reference.

### TECHNICAL FIELD

Embodiments of the present disclosure are directed towards the proper placement and verification of placement for patient physiological monitoring and therapeutic systems, such as cardiac devices.

### BACKGROUND

Patient physiological monitoring and therapeutic systems can be configured to measure physiological characteristics of a patient. Patient physiological monitoring systems include cardiac devices such as heart failure management systems that utilize radio frequency signals for the determination of a fluid level in the thoracic cavity. Part of the use of these cardiac device may be based on consistent placement on the patient's body.

### SUMMARY

Disclosed are systems and methods for the proper placement and verification of placement for patient physiological monitoring and therapeutic systems such as cardiac devices. In some implementations, the cardiac devices can include radio frequency (RF) based measurements for the management of heart failure.

In some aspects, the techniques described herein relate to an RF-based physiological monitoring device placement verification system, including: a patch configured to be adhesively coupled to skin of a patient; an RF sensor configured to be mechanically coupled to the patch, the RF sensor including at least one radio frequency (RF) antenna and associated transmitter/receiver circuitry configured to transmit RF waves into and receive reflected RF waves from a body of the patient; and at least one processor in electrical communication with the at least one radio frequency (RF) antenna and associated transmitter/receiver circuitry, the at least one processor configured to generate first one or more RF metric signals during a first period of time based on the transmitted and/or reflected RF waves associated with a prescribed anatomical location of the patch on the body of the patient, wherein the first one or more RF metric signals is configured for determining first one or more thoracic fluid parameters of the patient; generate second one or more RF metric signals during a second period of time subsequent to the first period of time based on the transmitted and/or reflected RF waves associated with a re-placement of the patch at a proposed anatomical location of the body of the patient, analyze the first and second one or more RF metric signals to determine a re-placement score associated with the re-placement of the patch at the proposed anatomical location of the body of the patient; determine whether the re-placement score transgresses a predetermined threshold value; and provide an alert indicating that the re-placement of the patch at the proposed anatomical location is acceptable if the placement score does not transgress the predetermined threshold value.

In some aspects, the techniques described herein relate to a system, wherein the mechanical coupling of the RF sensor to the patch is reversible.

In some aspects, the techniques described herein relate to a system, wherein the at least one processor is further configured to determine at least one of a cardiac output and a thoracic fluid value based on the transmitted and received RF waves.

In some aspects, the techniques described herein relate to a system, wherein the at least one processor is remote from the RF sensor.

In some aspects, the techniques described herein relate to a system, wherein the at least one radio frequency (RF) antenna and associated transmitter/receiver circuitry includes at least one transmitter and at least one receiver antenna.

In some aspects, the techniques described herein relate to a system, wherein the at least one radio frequency (RF) antenna and associated transmitter/receiver circuitry includes at least one antenna configured to transmit RF waves and receive reflected RF waves.

In some aspects, the techniques described herein relate to a system, wherein the alert is provided to a user device remote from and in communication with the at least one processor.

In some aspects, the techniques described herein relate to a system, wherein the alert includes an audible sound, a visual indicator, or a vibration.

In some aspects, the techniques described herein relate to a system, wherein determining a re-placement score includes comparing at least one of a phase and/or amplitude of one or more reflected RF waveforms associated with the first and second one or more RF metrics.

In some aspects, the techniques described herein relate to a system, wherein determining a re-placement score includes determining at least one of an average phase and/or average amplitude of reflected RF waveforms associated with the first and second one or more RF metrics.

In some aspects, the techniques described herein relate to a system, wherein determining a re-placement score includes comparing the at least one of a phase and/or amplitude of reflected RF waveforms associated with the first and second one or more RF metrics to a baseline phase and/or baseline amplitude, respectively.

In some aspects, the techniques described herein relate to a system, wherein the baseline phase and/or the baseline amplitude is determined based on one or more prior historical reflected RF waveforms for the patient and/or one or more populations of patients.

In some aspects, the techniques described herein relate to a system, wherein the predetermined threshold value is indicative of a phase and/or amplitude variation of 10 percent or less.

In some aspects, the techniques described herein relate to an RF-based physiological monitoring device placement verification system, including: a patch configured to be adhesively coupled to skin of a patient; an RF sensor configured to be mechanically coupled to the patch, the RF sensor including at least one radio frequency (RF) antenna and associated transmitter/receiver circuitry configured to be transmit RF waves into and receive reflected RF waves from a body of the patient; and at least one processor in electrical communication with the at least one radio frequency (RF) antenna and associated transmitter/receiver circuitry, the at least one processor configured to generate first one or more RF metric signals during a first period of time based on the transmitted and/or reflected RF waves associated with a prescribed anatomical location of the patch on the body of the patient, wherein the first one or more RF metrics is configured for determining first one or more thoracic fluid parameters of the patient, generate second one or more RF metric signals during a second period of time subsequent to the first period of time based on the transmitted and/or reflected RF waves associated with a re-placement of the patch at a proposed anatomical location of the body of the patient, analyze the first and second one or more RF metrics to determine a re-placement score associated with the re-placement of the patch at a proposed anatomical location of the body of the patient, determine whether the re-placement score transgresses a predetermined threshold value, and provide an alert indicating that the re-placement of the patch at the proposed anatomical location is unacceptable based on determining that the re-placement score transgresses the predetermined threshold value.

In some aspects, the techniques described herein relate to a system, wherein the mechanical coupling of the RF sensor to the patch is reversible.

In some aspects, the techniques described herein relate to a system, wherein the at least one processor is further configured to determine at least one of a cardiac output and a thoracic fluid value based on the transmitted and received RF waves.

In some aspects, the techniques described herein relate to a system, wherein the at least one processor is remote from the RF sensor.

In some aspects, the techniques described herein relate to a system, wherein the at least one radio frequency (RF) antenna and associated transmitter/receiver circuitry includes at least one transmitter and at least one receiver antenna.

In some aspects, the techniques described herein relate to a system, wherein the at least one radio frequency (RF) antenna and associated transmitter/receiver circuitry includes at least one antenna configured to transmit RF waves and receive reflected RF waves.

In some aspects, the techniques described herein relate to a system, wherein the alert is provided to a user device remote from and in communication with the at least one processor.

In some aspects, the techniques described herein relate to a system, wherein the alert includes an audible sound, a visual indicator, or a vibration.

In some aspects, the techniques described herein relate to a system, wherein determining a re-placement score includes comparing at least one of a phase and/or amplitude of one or more reflected RF waveforms associated with the first and second one or more RF metrics.

In some aspects, the techniques described herein relate to a system, wherein determining a re-placement score includes determining at least one of an average phase and/or average amplitude of reflected RF waveforms associated with the first and second one or more RF metrics.

In some aspects, the techniques described herein relate to a system, wherein determining a re-placement score includes comparing the at least one of a phase and/or amplitude of reflected RF waveforms associated with the first and second one or more RF metrics to a baseline phase and/or baseline amplitude, respectively.

In some aspects, the techniques described herein relate to a system, wherein the baseline phase and/or the baseline amplitude is determined based on one or more prior historical reflected RF waveforms for the patient and/or one or more populations of patients.

In some aspects, the techniques described herein relate to a system, wherein the predetermined threshold value is indicative of a phase and/or amplitude variation of 10 percent or less.

In some aspects, the techniques described herein relate to an RF-based physiological monitoring device placement verification system, including: a patch configured to be adhesively coupled to skin of a patient; an RF sensor configured to be mechanically coupled to the patch, the RF sensor including at least one radio frequency (RF) antenna and associated transmitter/receiver circuitry configured to be transmit RF waves into and receive reflected RF waves from a body of the patient; and at least one processor in electrical communication with the at least one radio frequency (RF) antenna and associated transmitter/receiver circuitry, the at least one processor configured to generate first one or more RF metric signals during a first period of time based on the transmitted and/or reflected RF waves associated with a prescribed anatomical location of the patch on the body of the patient, wherein the first one or more RF metrics is configured for determining first one or more thoracic fluid parameters of the patient, generate second one or more RF metric signals during a second period of time subsequent to the first period of time based on the transmitted and/or reflected RF waves associated with a re-placement of the patch at a proposed anatomical location of the body of the patient, analyze the first and second one or more RF metrics to determine a re-placement score associated with the re-placement of the patch at a proposed anatomical location of the body of the patient, determine whether the re-placement score transgresses a predetermined threshold value, and determine a correction responsive to determining the re-placement score transgresses a predetermined threshold value.

In some aspects, the techniques described herein relate to a system, wherein the mechanical coupling of the RF sensor to the patch is reversible.

In some aspects, the techniques described herein relate to a system, wherein the at least one processor is further configured to determine at least one of a cardiac output and a thoracic fluid value based on the transmitted and received RF waves.

In some aspects, the techniques described herein relate to a system, wherein the at least one processor is remote from the RF sensor.

In some aspects, the techniques described herein relate to a system, wherein the at least one radio frequency (RF) antenna and associated transmitter/receiver circuitry includes at least one transmitter and at least one receiver antenna.

In some aspects, the techniques described herein relate to a system, wherein the at least one radio frequency (RF) antenna and associated transmitter/receiver circuitry includes at least one antenna configured to transmit RF waves and receive reflected RF waves.

In some aspects, the techniques described herein relate to a system, wherein determining a re-placement score includes comparing at least one of a phase and/or amplitude of one or more reflected RF waveforms associated with the first and second one or more RF metrics.

In some aspects, the techniques described herein relate to a system, wherein determining a re-placement score includes determining at least one of an average phase and/or average amplitude of reflected RF waveforms associated with the first and second one or more RF metrics.

In some aspects, the techniques described herein relate to a system, wherein determining a re-placement score includes comparing the at least one of a phase and/or amplitude of reflected RF waveforms associated with the first and second one or more RF metrics to a baseline phase and/or baseline amplitude, respectively.

In some aspects, the techniques described herein relate to a system, wherein the baseline phase and/or the baseline amplitude is determined based on one or more prior historical reflected RF waveforms for the patient and/or one or more populations of patients.

In some aspects, the techniques described herein relate to a system, wherein the predetermined threshold value is indicative of a phase and/or amplitude variation of 10 percent or less.

In some aspects, the techniques described herein relate to a system, wherein third one or more RF metrics signals generated during a third period of time subsequent to the second period of time are generated based on applying the determined correction to the reflected RF waves.

In some aspects, the techniques described herein relate to a system, wherein the correction is for a thoracic fluid value.

In some aspects, the techniques described herein relate to a system, wherein the processor is configured to: determine if the determined correction is above a recalibration threshold; and perform a recalibration procedure when it is determined that the correction is above the recalibration threshold.

In some aspects, the techniques described herein relate to a system, wherein the recalibration threshold includes 10 percent or less of a maximum thoracic fluid value.

### BRIEF DESCRIPTION

In scenarios, various aspects of at least one example are discussed below with reference to the accompanying figures, which are not intended to be drawn to scale. The figures are included to provide an illustration and a further understanding of the various aspects and examples and are incorporated in and constitute a part of this specification but are not intended to limit the scope of the disclosure. The drawings, together with the remainder of the specification, explain principles and operations of the described and claimed aspects and examples. In the figures, each identical or nearly identical component that is illustrated in various figures is represented by a like numeral. For purposes of clarity, not every component may be labeled in every figure.

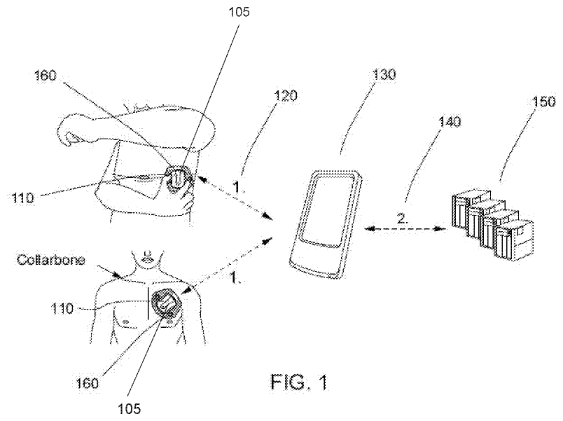

FIG. 1 shows an example schematic illustration of measurement and transmission of physiological data acquired via body-worn sensor(s) disclosed herein, according to some embodiments.

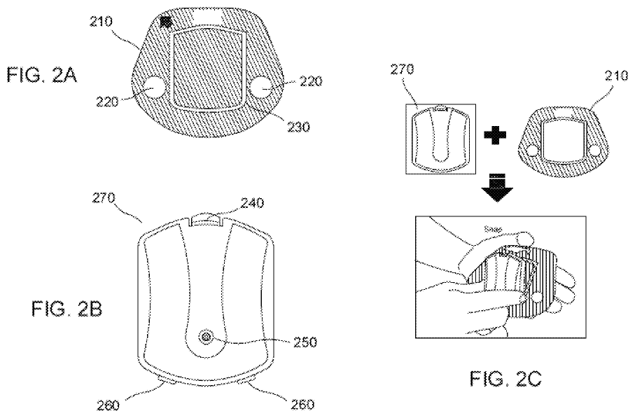

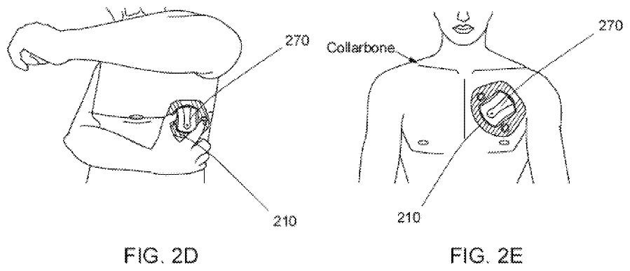

FIGS. 2A-E show an example sensor(s) disclosed herein, a patch configured to hold the sensor(s) in proximity to a body and attachment of a patch housing a sensor(s) onto skin of a patient, according to some embodiments.

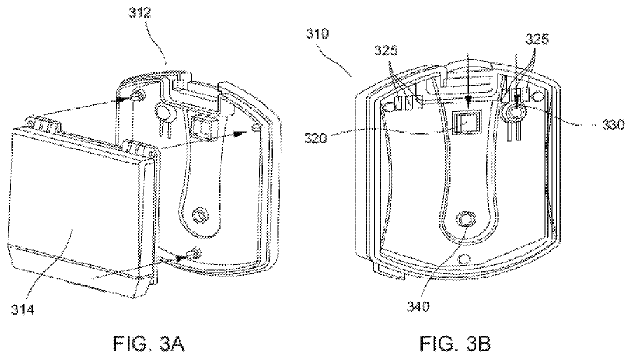

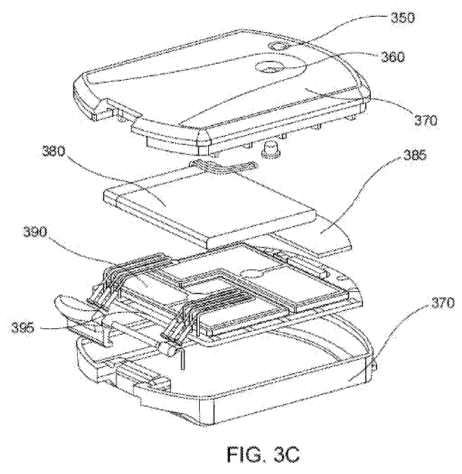

FIGS. 3A-C show example front, back and exploded views, respectively, of the sensor(s) disclosed herein, according to some embodiments.

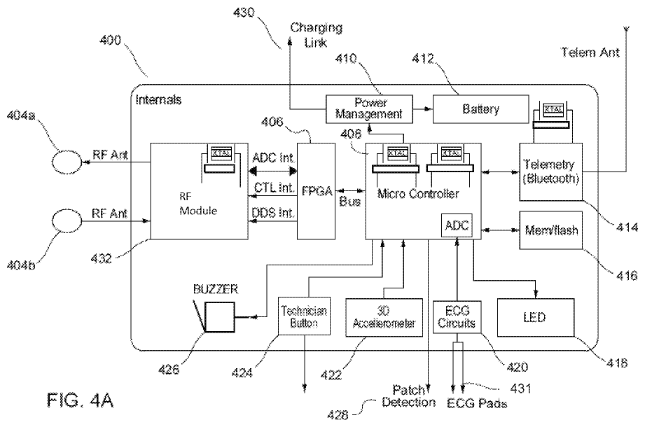

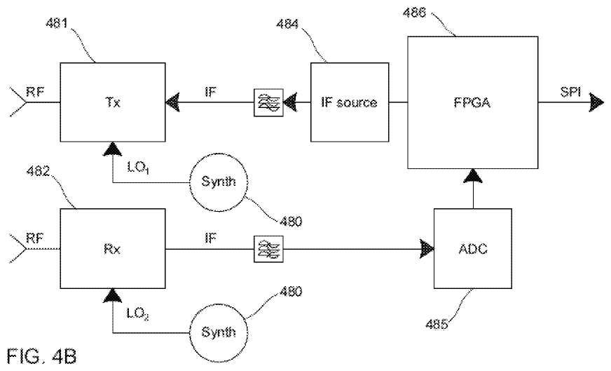

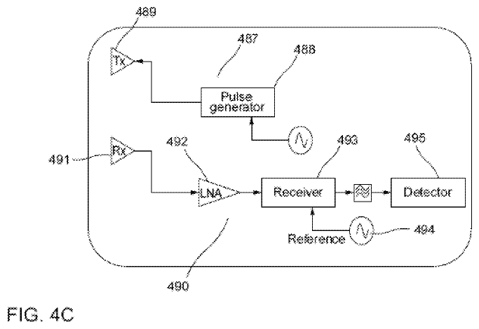

FIG. 4A shows an example illustration of device electronics architecture for measurements and transmission of patient physiological data, according to some embodiments.

FIG. 4B shows a block diagram of example architecture of a radio frequency (RF) module, according to some embodiments.

FIG. 4C shows a block diagram of another example architecture of an RF module, according to some embodiments.

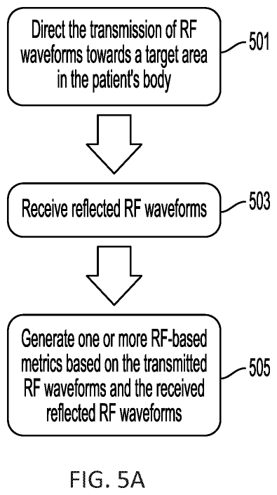

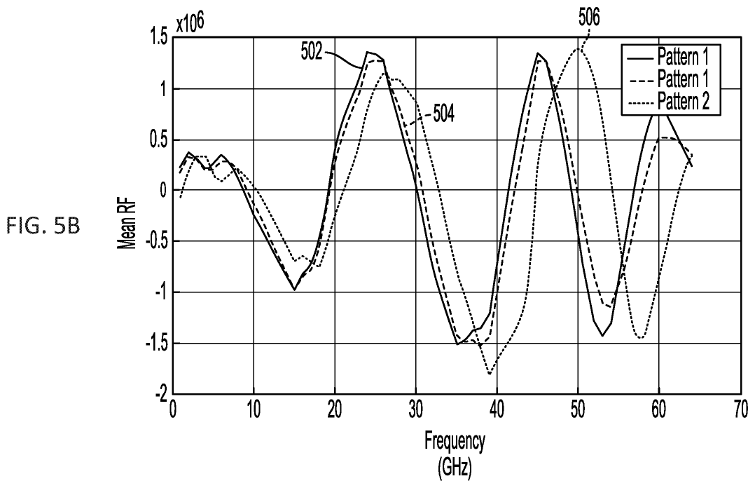

FIG. 5A illustrates a method of obtaining RF-based metrics using an RF-based physiological monitoring device, according to some embodiments.

FIG. 5B illustrates a method for comparing RF-based metrics obtained from an RF-based physiological monitoring device, according to some embodiments.

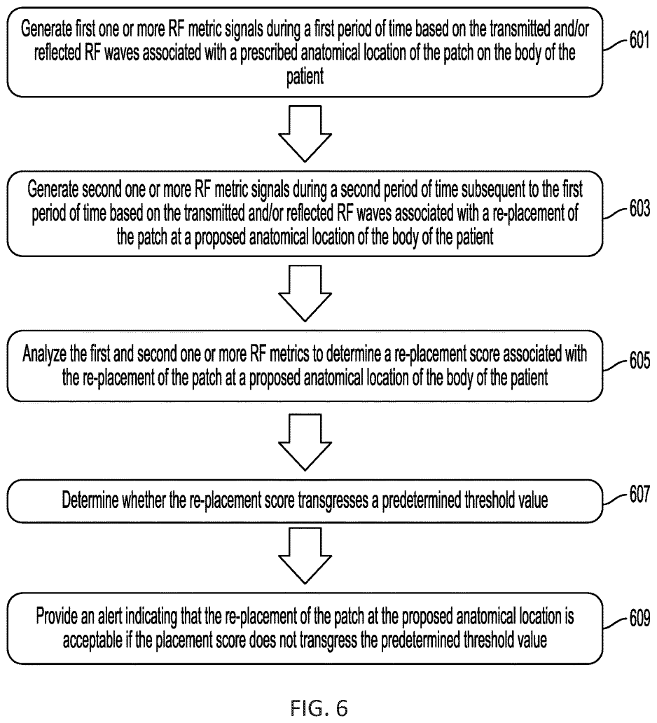

FIG. 6 illustrates a process or method for an RF-based physiological monitoring device placement verification system, according to some embodiments.

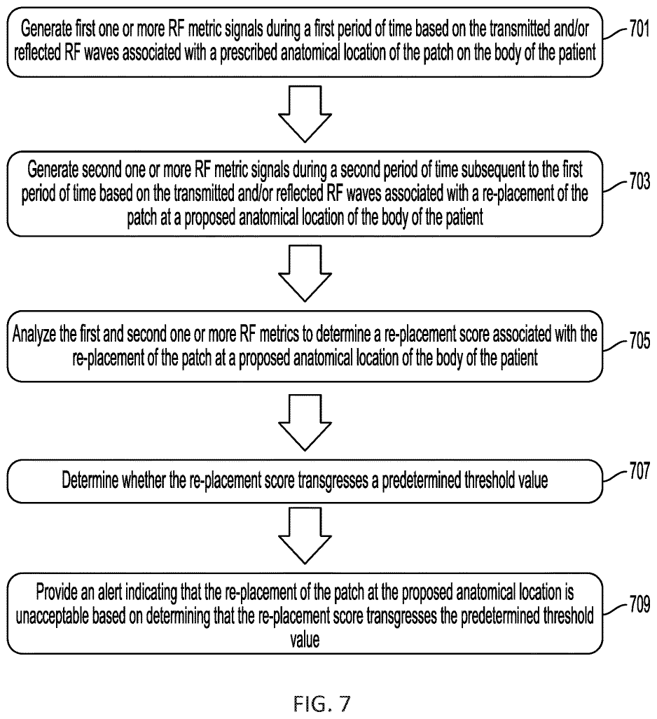

FIG. 7 illustrates a process or method for an RF-based physiological monitoring device placement verification system, according to some embodiments.

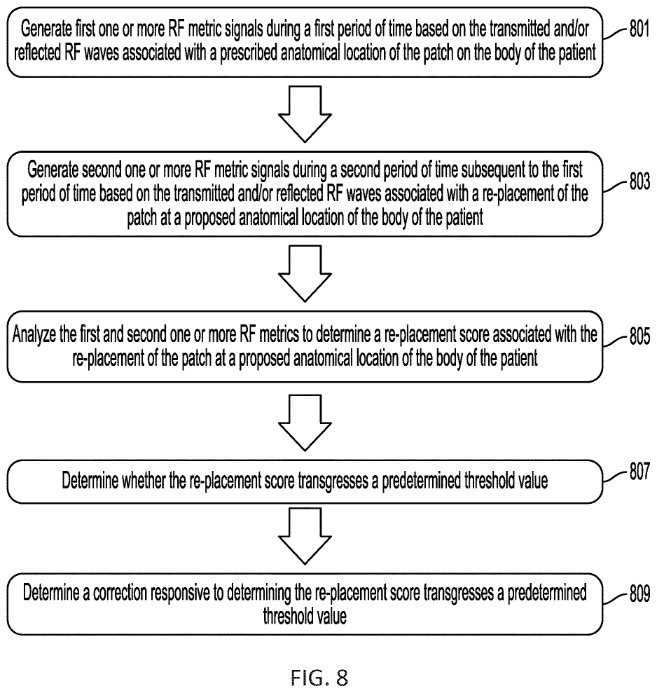

FIG. 8 illustrates a process or method for an RF-based physiological monitoring device with a correction feature, according to some embodiments.

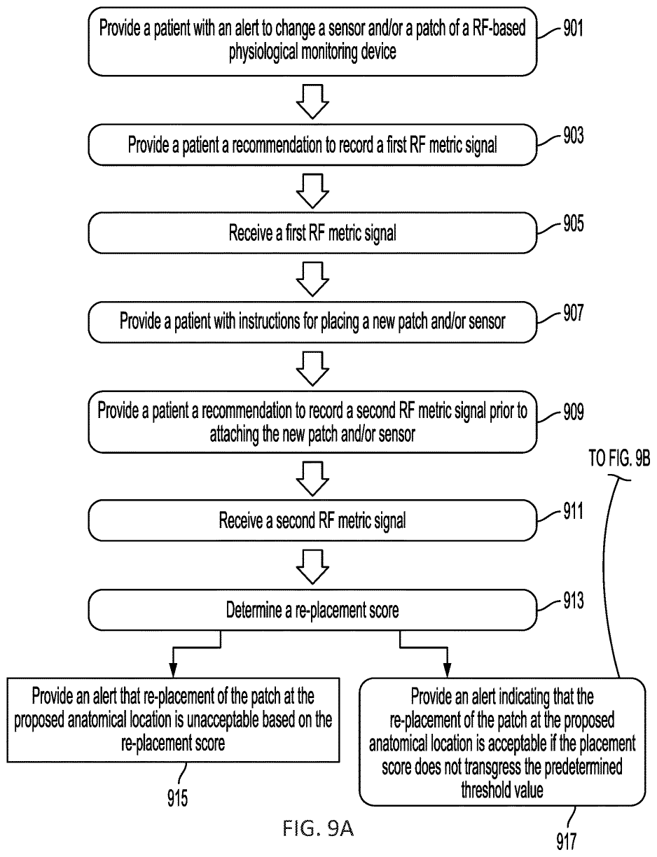

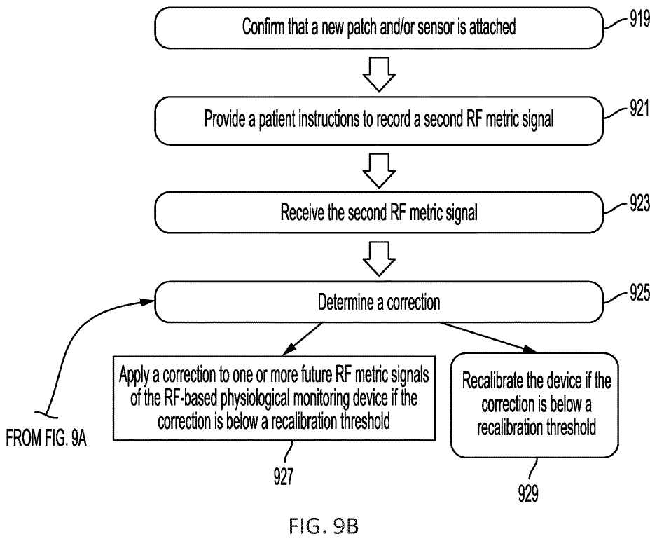

FIG. 9A. illustrates a process or method for an RF-based physiological monitoring device, according to some embodiments.

FIG. 9B illustrates a continuation of the process or method of FIG. 9A.

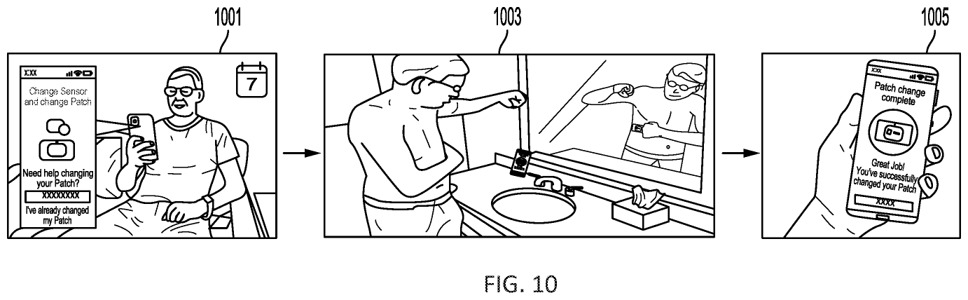

FIG. 10 illustrates graphical user interfaces for an RF-based physiological monitoring device, according to some embodiments.

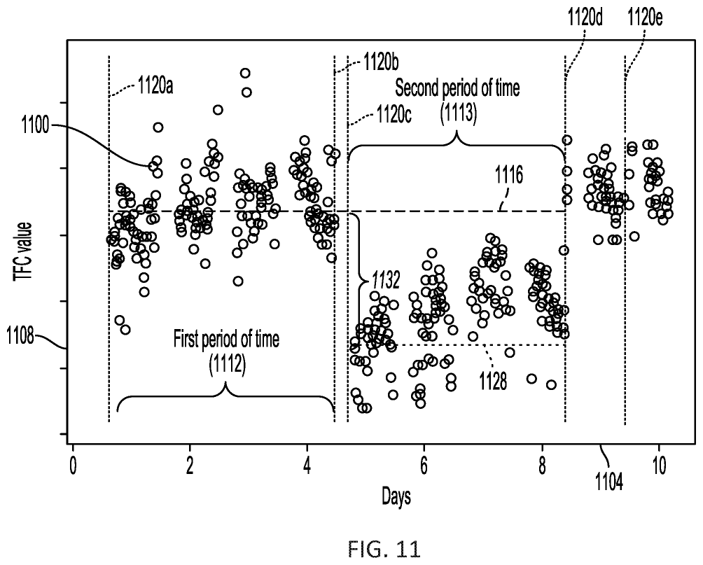

FIG. 11 illustrates experimental data in form of a scatter plot.

### DETAILED DESCRIPTION

Proper placement about the patient's body is beneficial for heart failure management systems, both to promote the correct functioning of cardiac devices configured to monitor heart failure and for the accuracy of long-term trends prepared from data gather by such cardiac devices. As patients wear such cardiac devices over long periods of time (e.g., days, weeks, months), patients often need to replace the cardiac device or components of the cardiac device and reattach the cardiac device to the same location on the body as the previously attached cardiac device. Changes in the positioning of the cardiac device on the body can lead to inaccurate readings or prohibit determining trends in the physiological parameters determined by the cardiac device.

Disclosed are systems and methods for the proper placement and verification of placement for patient physiological monitoring and therapeutic systems such as cardiac devices that utilize radio frequency (RF) based measurements for the management of heart failure. Accordingly, the disclosed systems and methods provide a way for improving the placement of a cardiac device when a patient re-attaches the cardiac device and verifying that the patient has accurately re-attached the cardiac device. Additionally, the system can be used to provide more accurate readings when a patient re-attaches the cardiac device in an improper position or slightly different location than the original placement of the cardiac device.

As discussed above, patient physiological monitoring and therapeutic systems can require the reattachment of devices such as cardiac devices to a prescribed anatomical location on the patient's body. The prescribed anatomical location may refer to a location on the patient's body where the device is placed to achieve accurate sensing of physiological characteristics. As these patient physiological monitoring and therapeutic systems are configured for continuous wear over long periods of time (e.g., days, weeks, or months) patients may desire or need to replace the devices or components of the device. For example, a patient may be instructed to replace a patch-based device every week and/or when an adhesive patch of the patch-based device starts to peel from the patient's body. In order to provide accurate readings and trending of physiological parameters determined by the devices, the devices or components of the device may need to be positioned and re-attached to the same prescribed anatomical location. Implementations herein include features to allow for devices to be re-attached by patients themselves, e.g., in proper position or attachment in a substantially same location as the original placement of the cardiac device. Implementations herein promote accuracy and allow for reliance in time-based trends of the parameters measured by the patient physiological monitoring and therapeutic systems.

Disclosed are systems, methods, and devices that are configured to enable the proper attachment or re-attachment of the device to a prescribed anatomical location. Additional systems and methods are disclosed that allow for more accurate determination of physiological parameters determined by the devices by applying a correction factor that accounts for drift or variance from the prescribed anatomical location.

In some embodiments, the cardiac device may be configured for a heart failure management system. For example, the cardiac device can include a non-invasive patch-based device that uses radio frequency (RF) technology for the early detection of changes in pulmonary fluid levels. An increase in pulmonary fluid levels is associated with heart failure decompression. Pulmonary fluid can increase over time and can be an early indicator of a heart failure decompensation event even prior to a patient experiencing noticeable symptoms. A non-invasive patch-based device can be configured for continuous 24-hour per day wear by a patient. Components of the non-invasive patch-based device can be water-resistant.

In some embodiments, the non-invasive patch-based device can utilize radio frequency (RF) technology to determine changes in pulmonary fluid levels and/or interstitial edema. For example, the device can include one or more RF antennas configured to emit and/or receive RF signals. RF signals can be emitted by the one or more RF antennas, propagate throughout the lungs and be reflected back to the one or more RF antennas. The reflected signals can be processed to determine various properties of body tissues located along the paths of the transmitted and/or scattered waves. For example, the reflected signals can be used to determine changes in pulmonary fluid levels and/or interstitial edema.

In some embodiments, the device can include additional sensors for monitoring and/or treatment. For example, the device can include one or more sensors for determining the heart rate, respiration rate, activity, posture, and heart rhythm (e.g., electrocardiogram (ECG)) of a patient.

Examples of RF-based cardiac devices for use in heart failure management are illustrated in FIGS. 1-4C. For example, the RF-based cardiac device illustrated in FIGS. 1-4C can be used for monitoring pulmonary fluid levels.

FIG. 1 depicts a fluid monitoring system that includes a physiological monitoring device 110, also hereinafter referred to as sensor(s), and a wearable patch 160 configured to place the sensor(s) on, or in the vicinity of, a surface of a body (e.g., a patient).

The fluid monitoring system as described herein can be implemented as a non-invasive, patch-based remote monitoring device. In example implementations, a primary measurement can be a Thoracic Fluid Index (TFI) or Thoracic Fluid Content (TFC) value. As described in further detail below, the fluid monitoring system is based on radiofrequency (RF) technology to detect changes in pulmonary fluid levels, a key early indicator of heart failure decompensation. The device emits radar waves that propagate through the lungs and reflect back to the sensor. Because these waves are modulated by tissue hydration, the system can detect interstitial edema (fluid buildup) days before a patient experiences physical symptoms like shortness of breath or swelling. The TFC value, in certain implementations, is used diagnostically as a relative, time-tracked measure of a patient's fluid status. In this regard, a higher TFC value can indicate increased fluid levels or increasing fluid levels trend, while a lower TFC can indicate decreased fluid level or decreasing fluid levels trend. In such manner, the fluid monitoring system can analyze patient-specific trends and provide alerts, notifications, or recommendations if, for example, fluid levels remain above a set threshold (e.g., for a period of time, such as three consecutive days). An alert allows a medical team to timely intervene. In implementations, in addition to fluid levels, the fluid monitoring system can record other physiological metrics to provide a comprehensive picture of patient health, such as heart rate and rhythm (e.g., based on ECG signals). In this manner, the fluid monitoring system can capture continuous cardiac data to monitor for arrhythmia or changes in heart rate. The fluid monitoring system can also monitor respiration rate. For example, the fluid monitoring system can measure the number of breaths per minute, track the patient's movement levels using a triaxial accelerometer, and/or track the patient's posture and sleep angle, e.g., by monitoring the patient's physical orientation. These metrics can help medical professionals monitoring the patient understand the patient's heart health because heart failure patients can experience increased difficulty breathing while lying flat (e.g., orthopnea).

As illustrated in FIG. 1, the wearable patch 160 and/or sensor(s) 110 can be configured to be worn at a prescribed anatomical location 105. For example, the sensor(s) 110 can be indicated to be positioned along the costo-clavicular space of a patient or along a side of the body. In examples, the sensor 110 is indicated to be positioned in prescribed anatomical locations that correspond to locations along the surface of the body that would provide an unobstructed path to the lung. Alternative locations that allow for the sensing of lung fluid levels with unobstructed access to the pleural space with less interference from organs, cartilage, tissue, nerves, vasculature, and/or bones can be used and is within the scope of this disclosure.

As an illustration, in some implementations, the sensor 110 can be indicated to be positioned approximately 2-3 inches under the armpit along approximately the mid-axillary line. In some examples, the sensor 110 can be indicated to be positioned approximately 0.5-4 inches under the armpit along approximately the mid-axillary line. In some examples, the sensor 110 can be indicated to be positioned approximately 0.5-6 inches under the armpit along approximately the mid-axillary line. In some examples, the sensor 110 can be indicated to be positioned approximately 0.5-8 inches under the armpit along approximately the mid-axillary line.

In some examples, the sensor 110 can be indicated to be positioned using substantially relative information or guidance. For instance, sensor 110 can be indicated to be positioned approximately 2 finger widths under the armpit along approximately the mid-axillary line. For instance, sensor 110 can be indicated to be positioned approximately 3 finger widths under the armpit along approximately the mid-axillary line. For instance, sensor 110 can be indicated to be positioned approximately 4 finger widths under the armpit along approximately the mid-axillary line.

Further, the wearable device including the sensor(s) 110 and wearable patch 160 described herein are configured for long-term and/or extended use or wear by, or attachment or connection to a patient. For example, devices as described herein may be capable of being used or worn by, or attached or connected to a patient, without substantial interruption, for example, up to 24 hours or beyond (e.g., weeks, months, or even years). In some implementations, such devices may be removed for a period of time before use, wear, attachment, or connection to the patient is resumed, e.g., to change batteries, carry out technical service, update the device software or firmware, and/or engage in other activities, without departing from the scope of the examples described herein. In some embodiments, the sensor(s) 110 and/or patch 160 can be removed and then re-applied or re-attached to the prescribed anatomical location in accordance with the examples described herein.

Data acquired by the sensor(s) 110 can be transmitted to a portable data transmission device (gateway) 130. The portable data transmission device (gateway) 130 can be capable of continuously transmitting data acquired by the sensor(s) 110 to one or more servers 150 for processing and/or analysis. Thus, for example, the gateway device 130 may transmit to the server 150 data received from the sensor(s) 110 with little or no delay or latency. To this end, in the context of data transmission between the device(s) 110 and server(s) 150, “continuously” for the present disclosure includes continuous (without interruption), or near continuous, i.e., within one minute after completion of a measurement by and/or an occurrence of an event on the device. Continuity may also be achieved by repetitive successive bursts of transmission, e.g., high-speed transmission. Similarly, the term “immediate,” according to the present disclosure, includes as occurring or done at once, or near immediate. For example, immediate may include within one minute after the completion of a measurement by and/or an occurrence of an event occurring on the device.

Further, in the context of physiological data acquisition by the device(s) 110, “continuously” also includes uninterrupted collection of sensor data, such as ECG data and/or accelerometer data, with clinical continuity. In this case, short interruptions in data acquisition of up to 1-second several times an hour or longer interruptions of a few minutes several times a day may be tolerated and can still be seen as “continuous”. As to latency as a result of such a continuous scheme as described herein, this relates to the overall budget of response time which can amount to between about 5 to about 15 minutes overall response time (e.g., time from when an event onset is detected to when a notification regarding the event is issued). As such, transmission/acquisition latency would therefore be in the order of minutes.

In some embodiments, the transmission of data/signals 120 between the sensor(s) 110 and the gateway device 130 may be a one way (e.g., from the sensor(s) 110 to the gateway device 130) or the transmission may be bi-directional. Similarly, the transmission of data/signals 140 between the gateway device 130 and the server 150 may be one way (e.g., from the gateway device 130 to the server 150) or bi-directional. The system may also include a charger (not shown) for powering the electronics of the system.

In some embodiments, the sensor(s) 110 is configured to monitor, record and transmit to the gateway device 130 physiological data about the wearer of the sensor(s) 110 continuously. In particular, the sensor(s) 110 may not interrupt monitoring and/or recording additional data while transmitting already acquired data to the gateway device 130. Put another way, in some embodiments, both the monitoring/recording and the transmission processes occur at the same time or at least nearly at the same time.

As another example, if the sensor(s) 110 does suspend monitoring and/or recording additional data while it is transmitting already acquired data to the gateway device 130, the sensor(s) 110 may then resume monitoring and/or recording additional data prior to all the already acquired data being transmitted to the gateway device 130. In other words, the interruption period for monitoring and/or recording may be less in comparison to the time it takes to transmit the already acquired data (e.g., between about 0% to about 80%, about 0% to about 60%, about 0% to about 40%, about 0% to about 20%, about 0% to about 10%, about 0% to about 5%, including values and subranges therebetween), facilitating the near-continuous monitoring and/or recording of additional data during transmission of already acquired physiological data. For example, in one specific scenario, when a measurement time duration is around 2 minutes, any period of suspension or interruption in the monitoring and/or recording of subsequent measurement data may range from a just few milliseconds to about a minute. Example reasons for such suspension or interruption of data may include allowing for the completion of certain data integrity and/or other on-line tests of previously acquired data as described in further detail below. If the previous measurement data has problems, the sensor(s) 110 can notify the patient and/or remote technician of the problems so that appropriate adjustments can be made.

In some embodiments, the bandwidth of the link 120 between the sensor 110 and the gateway device 130 may be larger, and in some instances significantly larger, than the bandwidth of the acquired data to be transmitted via the link 120 (e.g., burst transmission). Such embodiments ameliorate issues that may arise during link interruptions, periods of reduced/absent reception, etc. In some embodiments, when transmission is resumed after interruption, the resumption may be in the form of last-in-first-out (LIFO). The gateway device 130 can be configured to operate in a store and forward mode where the data received from the sensor 110 is first stored in an onboard memory of the gateway device and then forwarded to the external server 150. For example, such a mode can be useful where the link with the server 150 may be temporarily unavailable. In some embodiments, the gateway device 130 can function as a pipeline and pass through data from the sensor 110 immediately to the server 150. In further examples, the data from the sensor may be compressed using data compression techniques to reduce memory requirements as well as transmission times and power consumptions.

In some embodiments, the sensor(s) 110 may be configured to monitor, record, and transmit some data in a continuous or near-continuous manner as discussed above, while monitoring, recording, and transmitting some other data in a non-continuous manner (e.g., periodically, non-periodically, etc.). For example, the sensor(s) 110 may be configured to record and transmit electrocardiogram (ECG) data continuously or nearly continuously while radio frequency (RF) based measurements and/or transmissions may be periodic. For example, ECG data may be transmitted to the gateway device 130 (and subsequently the server 150) continuously or near-continuously as additional ECG data is being recorded, while RF-based measurements may be transmitted once the measuring process is completed.

Monitoring and/or recording of physiological data by the sensor(s) 110 may be periodic, and in some embodiments, may be accomplished as scheduled (e.g., periodically) without delay or latency during the transmission of already acquired data to the gateway device 130. For example, the sensor(s) 110 may acquire physiological data from the patient (i.e., the wearer of the sensor(s) 110) in a periodic manner and transmit the data to the gateway device 130 in a continuous manner as described above.

The sensor(s) 110 may be configured to transmit the acquired data to the servers 150 instead of, or in addition to, transmitting the data to the gateway device 130. The sensor(s) 110 may also be configured to store some or all of the acquired physiological data. In some embodiments, the transmission of data from the sensor(s) 110 to the gateway device 130 may be accomplished wirelessly (e.g., Bluetooth®, etc.) and/or via a wired connection, e.g., as shown by transmission link 120. The transmission of data from the gateway device 130 to the server 150 may also be accomplished wirelessly (e.g., Bluetooth®-to-TCP/IP access point communication, Wi-Fi®, cellular, etc.) and/or via a wired connection, e.g., as shown by transmission link 140.

As mentioned above, in some embodiments, the transmission of data and/or signals occurs via two links 120, 140, the links between the sensor(s) 110 and the gateway device 130 (e.g., Bluetooth® link) and between the gateway device 130 and the server 150 (e.g., Wi-Fi®, cellular, and/or the like). The Bluetooth® link can be a connection bus for sensor(s) 110 and server 150 communication, used for passing commands, information on status of the microprocessor of the sensor(s) 110, measurement data, etc. In some embodiments, the microprocessor of the sensor(s) 110 may initiate communication with the server 150 (and/or the gateway device 130), and once connection is established, the server 150 may be configured to initiate some or all other communications. In some embodiments, the gateway device 130 may be configured to conserve the power available to the sensor(s) 110, device 130 and/or servers 150. For example, one or both links 120, 140 may enter power saving mode (e.g., sleep mode, off-state, etc.) when the connections between the respective devices/server are not available. As another example, the transmission of data may also be at least temporarily interrupted when the link quality (e.g., available bandwidth) is insufficient for at least a satisfactory transmission of the data. In such embodiments, the gateway device 130 may serve as a master device in its relationship to one or both of the sensor(s) 110 and the server 150.

In some embodiments, the gateway device 130 may be considered as a simple pipe, the sensor-gateway device-server path may be defined as a single link. For example, the link performance may depend on the bottleneck between the sensor-gateway device and gateway device-server links 120, 140. In some embodiments, at least the main bottleneck may be the gateway device-server link 140, since the gateway device is carried by the patient in close proximity to the device, while the gateway device-server link 140 (e.g., cellular or Wi-Fi® coverage, and/or the like) is expected to be variable. In some embodiments, a “best effort delivery” quality-of-service may be sufficient for the Bluetooth link and/or the TCP/IP link, since the transmitted data is processed (with some latency, for example) and is used for displaying notifications (for example, instead of being presented online to a monitoring center). In some embodiments, a single gateway device 130 may be configured to serve a plurality of sensors. For example, the plurality of sensors may be connected to a single gateway device 130 via respective links. In some embodiments, there may be a plurality of gateway devices serving one or more sensor(s). For example, each sensor of one or more sensors may be connected to a plurality of gateway devices via respective links.

In some embodiments, the transmission links 120, 140 may be configured to withstand co-existence interference from similar devices in the vicinity and from other devices using the same RF band (e.g., Bluetooth®, Cellular, Wi-Fi®, and/or the like). Standard Bluetooth® protocol and/or standard TCP/IP protocols, as well as the addition of cyclic redundancy check to the transmitted data may be used to address any issue of interference. Further, to preserve the security of wireless signals and data, in some embodiments, data transfer between the sensor and the server may be done using a proprietary protocol. For example, TCP/IP link may use SSL protocol to maintain security, and the Bluetooth® link may be encrypted. As another example, UDP/HTTP may also be used for secure transmission of data. In some embodiments, only raw binary data may be sent, without any patient identification.

Examples of the types of physiological data that the arrhythmia and fluid monitoring sensor(s) 110 is configured to monitor and/or acquire from a patient wearing the sensor(s) 110 include one or more of electrocardiogram (ECG) data, thoracic impedance, heart rate, respiration rate, physical activity (e.g., movement), and patient posture. In some embodiments, the physiological data may be acquired and/or transmitted to the gateway device 130 or the server 150 by the sensor(s) 110 in a manner that is continuous, periodic, or as instructed by received signals (e.g., as instructed by signal received from the gateway device 130 and/or the server 150). For example, the wearer of the sensor or another party (e.g., a health professional) may activate the sensor(s) 110 and the sensor 110 may start monitoring and/or recording any one of the above-noted physiological parameters automatically without further input from the wearer or the party. The sensor(s) 110, or the arrhythmia and fluid monitoring system in general, may request further input (e.g., selection of a setting identifying the physiological parameter to be measured) before initiating the monitoring and/or recording of physiological data. In any case, once the monitoring and/or recording starts, the sensor(s) 110 may transmit the acquired data to the gateway device 130 and/or the server 150 in an at least a continuous manner as described above, for example.

In some embodiments, one or more of the above-noted physiological parameters may be measured periodically, and the sensor(s) 110 may transmit the measurements to the gateway device 130 in an at least a continuous manner as acquired. For example, the periodic measurements may proceed as scheduled and the transmission to the gateway device 130 may occur with little or no delay or latency after data is acquired.

In some embodiments, the sensor(s) 110, or the fluid monitoring system in general, may be configured to operate some, but not all, of the available features discussed above. For example, the sensors 110 may be configured to monitor and/or acquire one or more of ECG data, thoracic impedance, heart rate, respiration rate, physical activity (e.g., movement), patient posture, etc., but not the others. For instance, the sensors may be configured to monitor and/or acquire data such as ECG data, but not respiration rate, physical activity (e.g., movement), patient posture. Such embodiments may be affected, for example, by including controls in the sensors and/or the system that separately control components of the sensors/system responsible for the features. For example, the fluid monitoring system may include controls (e.g., power buttons) that separately control the accelerometer and the ECG components of the sensor. By switching on the accelerometer power control and switching off the ECG power control, in some embodiments, one may allow the monitoring and/or acquiring of data related to respiration rate, physical activity, and patient posture while deactivating the monitoring and/or acquiring of ECG data.

In some embodiments, an adhesive patch 160 may be used to attach the sensor(s) 110 to a surface of the body of a patient.

FIGS. 2A-E show the sensor 270 disclosed herein (analogous to sensor(s) 110), and a patch 210 (analogous to patch 160) configured to attach the sensor 270 to a patient's body or at least hold the sensor 270 in proximity to skin of the body. As shown in FIGS. 2A-2E, the sensor 270 can be removably attached to the patch 210.

The patch 210 may include a patch frame 230 (e.g., plastic frame) delineating the boundary of the region of the patch 210 that is configured for housing the sensor 270. The patch 210 may be disposable (e.g., single- or few-use patches), and may be made of biocompatible, non-woven material. In some embodiments, the sensor 270 may be designed for long-term usage. In such embodiments, the connection between the patch 210 and the sensor 270 may be configured to be reversible. To illustrate, the sensor 270 may be configured to be removably attached to the patch 210. For example, the sensor 270 may include components such as snap-in clips 240 that are configured to secure the sensor 270 to the patch 210 (e.g., the patch frame 230) upon attachment (and released the sensor 270 from the patch when separation is desired). The sensor 270 may also include positioning tabs 260 that facilitate the attachment process between the sensor 270 and the patch 210. In some embodiments, the patch may be designed to maintain attachment to skin of a patient for several days (e.g., in the range from about 4 days to about 10 days, from about 5 days to about 7 days, and/or the like). In implementations, the sensor 270 may include one or more indicators 250 configured to provide communications to the patient. For example, the one or more indicators 250 may include an LED configured to light up green when the patient has placed the patch 210 at the correct anatomical location 250 and red when the patient has placed the patch 210 at an incorrect anatomical location.

In some embodiments, the patch 210 may include additional components that facilitate or aid with the monitoring and/or recording or acquiring of physiological data by the sensor 270. For example, the patch may include conductive elements such as one or more ECG electrodes 220 (e.g., a single lead, two leads, etc.) that can be used when recording ECG data from the surface (e.g., skin contacted directly or through a covering) of a patient's body. The electrodes may be coupled to the sensor 270 by dedicated wiring within the patch. In some embodiments, the ECG may have a sampling rate in the range from about 250 Hz to about 500 Hz, from about 300 Hz to about 450 Hz, from about 350 Hz to about 400 Hz, including values and subranges therebetween. In some embodiments, the ECG signal may be sampled after band-pass filtering by a 12 bit analog-to-digital converter (ADC). During normal operation, data may be transferred to the server “as-is” and can then be used by the server algorithms for analysis. In some embodiments, an internal algorithm allows for real-time evaluation of the ECG signal quality upon each attachment of the device to the patient (“attachment test”).

Examples of locations on the surface of a patient body at which a patch may be placed are shown in FIGS. 2D-E, where a patch 210 housing sensor 270 is shown as placed at on the side (below armpit, for example) (FIG. 2D) and upper chest (FIG. 2E) of the torso of a patient. The patch 210 can be placed at the prescribed anatomical location. It is to be noted that the patch may be placed on any part of the surface of a patient's body that allows for efficient monitoring and recording of a physiological data (e.g., area of skin that allows for uniform attachment of the patch 210 to the skin). For example, one may place the patch 210 under an armpit at the nipple level for performing thoracic fluid level measurements. With respect to ECG measurements, the ECG signal at this location may be represented as the difference between standard V5 and V6 leads of an ECG.

With reference to FIGS. 3A-C, in some embodiments, front, back and exploded views, respectively, of the sensor(s) disclosed herein are shown. FIG. 3A shows the front 312 and back 314 covers of the sensor 310 (labelled as top and bottom covers 370 in FIG. 3C). In some embodiments, such covers may couple to each other to seal the electrical components of the sensor from the surrounding environment (e.g., electrical sealing). In such embodiments, metallic tabs 325 may protrude outside the covers to provide electrical connection for situations such as performing ECG measurements, charging power source and/or the like.

FIG. 3B shows that the sensor 310 may include one or more indicators that identify the status of the sensor 310 to the user of the sensor 310. Examples of such indicators include but are not limited to light indicator 340 (e.g., a light emitting diode (LED) indicator) and sound indicators 320. In some embodiments, the indicators 320, 340 provide feedback on the status of the sensor 310 and components thereof, such as the charging and/or power level of the power source of the sensor 310 (e.g., a battery), the attachment level of the sensor 310 to the patch 210, the attachment level of the patch 210 to the surface of the body to which the patch 210 is attached, etc. As another example, the sensor may respond by blinking (e.g., via the light indicator 340) or buzzing (e.g., via the sound indicator 320) in response to an engagement by a patient to indicate possible symptoms.

In some embodiments, FIG. 3C provides an exploded view of the sensor 310 depicting at least some of the components of the sensor. For example, the sensor 310 may comprise a power source such as a battery 380, a light indicator 360, a button 350 for facilitating the interaction (e.g., of a patient, a healthcare provider, and/or a technician with the sensor 310), a wireless communications circuit 385, a radio frequency shield 390 (such as a metallic cover, e.g., to prevent interferences with the ECG processing and other digital circuitry), a digital circuitry board 395, and/or the like. FIG. 3C shows a Bluetooth unit as an example of a wireless communications circuit 385, although in addition to or alternatively to the Bluetooth unit, other modules facilitating other types of communications (examples of which including Wi-Fi®, cellular, and/or the like) may be included in the sensor 310.

In some embodiments, the sensor 310 may also include input interfaces such as buttons for interfacing with a user. For example, the sensor may include a button 330 that allows a patient or a health care professional to activate or deactivate the sensor 310. Such input interfaces may be configured to avoid or at least minimize unintended interactions with a user. For example, a button may be sized and shaped to avoid accidental activation (e.g., the button may be configured to require activation by being pushed in with an external object). This button may be used to reset the sensor as well as pair the sensor to the gateway device and initiate communication. In some embodiments, the input interface of the sensor may include a touch screen configured to receive input from a user and/or provide information back to the user. For example, the input may allow the user to set the sensor in an “airplane mode,” i.e., for example by deactivating any wireless communication (e.g., Wi-Fi®, Bluetooth®, etc.) with external devices and/or servers. For example, the button can be implemented as a magnetic switch, e.g., an embedded magnetic switch, instead of a physical button. Such an implementation can be useful for designing the housing of the device and avoid exposing button components to the environment.

In some embodiments, as described above, the disclosed sensor is configured to monitor and/or acquire data on physiological parameters including but not limited to electrocardiogram (ECG) data, thoracic impedance, heart rate, respiration rate, physical activity, posture, and/or the like. To that effect, the sensor and/or the patch housing the sensor may include components that facilitate or undertake the monitoring and/or recording of at least some of these parameters. For example, as noted above, the patch housing the sensor may include ECG electrodes coupled to the sensors to facilitate the monitoring and/or acquiring of ECG data. As shown in FIG. 4A, which shows an example embodiment of device electronics architecture for measurements and transmission of patient physiological data, the sensor includes EGG processing circuitry 420 configured to couple to the ECG electrodes embedded in the patch housing the sensor itself. The ECG processing circuitry 420 is configured to, for example, perform filtering, amplification, and/or removal of noise, low frequency variations in the signal, and other signal artifacts.

As another example, the sensor may include at least one radio frequency (RF) antenna for directing electromagnetic waves into a body of a patient and receiving waves that are scattered and/or reflected from internal tissues. Further, the sensor may include RF circuitry or module configured to process the received waves so as to determine some properties of the tissues that are on the path of the transmitted and/or scattered/reflected waves. For example, the at least one antenna may direct RF waves towards a lung of a patient and the RF circuitry may analyze the scattered/reflected waves to perform an RF-based measurement of the thoracic fluid level of the patient. FIG. 4A shows an example embodiment of a sensor comprising RF antennas, an RF module, and circuits for controlling the module (e.g., field-programmable gate array (FPGA) circuits).

With reference to FIG. 4A, in some embodiments, the sensor 400 includes external interfaces such as but not limited to RF antennas (e.g., bi-static) 404a, 404b for transmitting & receiving RF signals, a button or switch 424 for activating or deactivating the sensor 400, an LED 418 and a buzzer 426 for providing light and audio feedback to a user of the sensor 400, and/or a battery charging link 430 coupled to a power management module 410 for charging an onboard power source such as a battery 412. In implementations, the external interfaces may be able to electrically connect the sensor 400 to additional functionalities. For example, the sensor 400 may include links connecting the sensor 400 to an adhesive patch containing ECG electrodes such that the links provide a connection to ECG pads 431 and for patch detection 428. In implementations, these links may be the same links, for example, that provide the charging link 430. In some embodiments, the sensor 400 may also include a wireless link (e.g., Bluetooth®) (not shown) to provide an external server access to the sensor 400 so as to exert at least some control on the sensor 400. The wireless link may occur, for example, using telemetry 414, such as a Bluetooth® module.

Internally, in some embodiments, the sensor 400 may include a microprocessor 408 (which may be alternatively referred to as a micro-controller) that includes instructions thereon specifying how measurements (e.g., RF, ECG, accelerometer, etc.) are taken and the obtained data are transmitted, how to relay the status of the sensor 400, how/when the sensor 400 can enter the plurality of sleep levels, and/or the like. In some embodiments, the instructions may also specify the conditions for performing certain types of measurements. As another example, the instructions may identify the conditions that may have to be fulfilled before ECG measurements can commence, such conditions including at least sufficient attachment level between the sensor and the surface on the body to which the sensor 400 is attached. As another example, the instructions may specify that the RF measurements cannot be taken unless data from the accelerometer indicates that the patient is stable (e.g., at rest and/or holding a certain posture). In some embodiments, the microprocessor 408 may have internal and external non-volatile memory banks 416 that can be used for keeping measurement directory and data, scheduler information, and/or a log of actions and errors. This non-volatile memory allows saving power via a total power-down while retaining data and status information.

FIGS. 4B and 4C are block diagrams that illustrate examples of RF sensor functionality disposed within an RF module (e.g., RF module 432) according to some embodiments. As noted herein, such functionality may be used for RF based fluid monitoring of fluid accumulation/content in tissue in accordance with the techniques described herein. Referring first to FIG. 4B, initially, one or more RF signals (e.g., a single “LO” signal, or different “LO1” and “LO2” signals, collectively “LO” signals) can be generated by a broadband synthesizer 480 (e.g., a pulse generator and synthesizer-LO). Such a synthesizer 480 can preferably include moderate phase noise performance and/or fast settling time capabilities (e.g., in some embodiments, one or the other). The RF module includes a transmitter portion 481, including a transmitting antenna (Tx) and associated circuitry for transmitting RF waves directed, for example, towards a tissue of interest in the patient's body, and a receiver portion 482, including a receiver antenna (Rx) and associated circuitry 482 for receiving reflected RF waves from, for example, the tissue of interest in the patient's body.

The LO signal at the transceiver (Tx) of the transmitter portion 481 is multiplied with an external sine wave at a low frequency intermediate frequency (IF) signal, generated by an IF source 484, and directed to the output of the transceiver (Tx). As noted above, the LO signal at transceiver portion 481 and the receiver portion 482 can be generated by one or two LO sources (e.g., synthesizer(s) 480). Output power can be controlled via digital control of a digitally controlled attenuator (DCA) on the RF transceiver path. An external reflected RF wave returning to a receiving antenna (Rx) is directed to the receiver portion and down-converted to an IF frequency by a down conversion mixer. The reflection characteristics (phase and amplitude) can be transformed to a new IF carrier (e.g., on the order of 250 kHz), filtered and amplified before the ADC 485.

Digital control for the functionality in FIG. 4B may be achieved directly by a processor and/or digital logic (e.g., an FPGA 486), which may be configured to control both the transceiver's configuration process, IF signal adjustments and associated switching.

Referring now to FIG. 4C, in some embodiments, the RF module 432 may be implemented using a transmitting portion 487 and receiver portion 490 as shown. For example, the transmitter portion 481 can include a pulse generator 488 and a transmitting antenna Tx 489 for transmitting the RF waves directed towards a tissue of interest in the patient's body. The receiver portion 490 may include a receiving antenna Rx 491, a low-noise RF amplifier 492, a receiver 493 that converts the reflected RF signals to an IF signal by using mixer and local oscillator 494, which may be a monostatic (sheared LO) or a bi-static system. The signal can be filtered, amplified, and fed into a detector 495, the output of which may be connected to additional circuitry for further signal processing.

With respect to potential RF/ECG interference, in some embodiments the following steps can be taken: (1) Ground Separation between digital and RF components may be achieved by separating the digital and RF grounds, and utilizing a single connection point through ferrite bead; (2) RF module shielding may also be used which may comprise a metallic cover, for example, radio frequency shield 390 as shown in FIG. 3C; (3) Power circuitry considerations can include different power paths which may be utilized for different components/modules; additionally, the power circuit may include filters to avoid noise; (4) ECG filtering may also be used to aid in minimizing RF interference which prevents high frequency signals interfering with the ECG circuitry/module; (5) Circuitry layout can include ECG signal paths that are physically separated from RF paths, and/or from other lines that might interfere.

FIG. 4C shows an example general architecture of the RF module with low frequency IF and shared local oscillator (LO). As an example, non-limiting example, with reference to FIG. 4C, the transmitted RF signal may be mixed with the IF signal (e.g., about 250 kHz) before transmission, so the transmission is actually two (2) tones around the carrier RF signal, separated by about 500 kHz.

In some embodiments, the RF module 432 may include a calibration path (e.g., an electric reflector such as but not limited to a resistor on board) which generates a steady and constant or near-constant reflection uncorrelated with the external propagation path. This reflector generates a reflection profile with minimal dependencies to temperature, system noise, and device location on the body.

In some embodiments, the RF module 432 itself may not have any processing components inside. For example, it may be controlled by a field-programmable gate array (FPGA) that defines in each or nearly each frequency point one or more of the frequency, output power levels, system gain, bypassing modes, and/or enable/disable transmissions.

In some embodiments, the RF module 432 may support different types of waveform configurable options, including but not limited to normal operation, calibration frame operation, interleaved switching between normal and calibration frame operation, interleaved switching between normal and delayed path operation, and/or clear channel sensing. In some of these options, for example the normal and interleaved switching ones, the attenuation may be different per frequency, while in the case of clear channel sensing, there may not be any transmission. For the calibration frame operation, the attenuation can be the same for all frequencies but may be higher when compared to those of the normal operation.

In some embodiments, the transmit (Tx) and receive (Rx) switches may be respectively set to transmit and receive through a calibration path for the case of calibration frame operation, while for the clear channel sensing, Rx switch may be set to antenna and Tx to calibration path. For interleaved switching between normal and calibration frame operations and between normal and delayed path operations, in some embodiments, the Tx and Rx switches may alternate between calibration and antenna path per frequency, and normal and delayed path, respectively.

In some embodiments, the RF waves may be in the frequency ranges from about 100 MHz to about 1 GHz, 200 MHz to about 2.5 GHz, from about 200 MHz to about 3 GHz, from about 500 MHz to about 5 GHZ, including values and subranges therebetween. In some embodiments, a thoracic fluid content (TFC) sensitivity may be configured to allow measurement of heart signals at distances up to about 25 cm, about 20 cm, about 15 cm, about 10 cm, about 5 cm, including values and subranges therebetween, inside the body onto which the disclosed sensor is attached. In some embodiments, the dynamic range is no less than 100 dB, measured in the presence of a strong coupling signal between transmission & reception. Further the waveform may be stepped frequency (e.g., 16-128 frequencies), arbitrary with 1 MHz accuracy and resolution. In some embodiments, actual frequencies selected may be contiguous or not, depending on regulatory requirements. In some embodiments, the dwell and settling times may be configurable to allow, for example, 16-128 frequencies within less than 5 to 20 ms, respectively.

In some embodiments, the FPGA 406, with a top-level view of which shown in FIG. 4D, may be configured to interface with the RF module 432. For example, the FPGA 406 is configured to one or more of control the transceiver processes, control the RF discrete pins, control the ADC processes, generate the IF signal for the RF module 432, and/or acquire ADC (analog-digital conversion) output samples, synchronized with the generated IF signal. Further, in some embodiments, the FPGA 406 is configured to process the ADC output samples to generate the baseband data. In addition, in some embodiments, the FPGA 406 may be configured to interface with the microcontroller or microprocessor 408. For example, the FPGA 406 may start RF transmission (per frame) upon command from microprocessor 408, save baseband data to local RAM, per frame, for microprocessor 408 to read, allow microprocessor 408 read/write transactions towards configuration memory, provide a debug interface for the microprocessor 408, and/or allow microprocessor 408 to change configuration settings using a dedicated memory.

In some embodiments, the FPGA can support up to 128 frequencies, allowing for a different gain and dwell time per frequency. In some embodiments, power consumption can be minimized by using several clock frequencies within the design and gating unused clock signals. In some embodiments, microprocessor data acquisition can be performed using a separate clock, allowing the shut-down of the entire control and processing pipe while reading the data.

In some embodiments, the sensor disclosed herein may comprise an accelerometer and the accelerometer may be used to determine one or more of the physical activity, posture and respiration rate of a patient wearing the sensor. For example, a three-axis (3D) accelerometer 422 may be used to acquire data on patient movements and posture as well as the respiration rate, and a processor (of the sensor or an external server, for example) receiving the acquired data may use the data (e.g., in conjunction with data obtained by the sensor such as ECG data or RF-based measurements) to determined physiological parameters of the patient, such as the thoracic fluid level of the patient. The 3D accelerometer 422 may be used to aid RF and/or ECG analysis by detecting different types of motion segments in the recording so that the conditions of the measurements of the RF and/or the ECG may be interpreted/analyzed accordingly. For example, in some embodiments, RF and/or ECG measurements may be performed while the patient wearing the sensor is active or at rest. The analysis of the RF and/or ECG data may then depend on the state of the patient's physical activity (e.g., at rest, low intensity activity, high intensity activity, etc.). In such embodiments, the accelerator may be used to identify the patient's physical state so as to properly analyze and interpret the RF and/or ECG measurements. In some embodiments, the accelerometer 422 may also contain an internal tap detector, which may be used for generating a patient triggered event (e.g., using “double tap” feature). The acceleration signal can be used to calculate respiration rate. For example, a processor (e.g., of the sensor 400 and/or a remote server) may filter high-frequency accelerometer data from low-frequency accelerometer data. Using the high-frequency accelerometer data, the processor may determine the patient's motion, and using the low-frequency accelerometer data, the processor may determine that patient's respiration.

FIG. 4A shows an example embodiment of a sensor comprising a 3D accelerometer 422, RF antennas 404a, 404b, ECG processing circuitry coupled to ECG electrodes, a microcontroller 408 (which may be alternatively referred as microprocessor throughout this disclosure) and a telemetry (e.g., Bluetooth®) 414. In such embodiments, for example, the micro-controller 408 may receive data on patient respiration rate, movements, posture, and/or ECG, as well as RF-based measurements of the patient, and process, and/or transmit to an external processor via the telemetry 414 for further processing, to determine a physiological parameter of the patient. As an example, the micro-controller 408 of the sensor may cause the Bluetooth® telemetry 414 to transmit the noted data and measurements to an external server which in turn analyzes the RF measurements, the ECG, posture, movement, and/or respiration rate data to determine the thoracic fluid level of the patient.

As another example, the external server may analyze ECG data to determine patient health conditions related to one or more of a heart rate, atrial fibrillation, flutter, supraventricular tachycardia, ventricular tachycardia, pause, atrioventricular (AV) block, ventricular fibrillation, bigeminy, trigemini, ventricular ectopic beats, supraventricular ectopic beats (SVEB), bradycardia, and/or tachycardia. The determination of patient physiological health parameters (e.g., thoracic fluid level or the above-noted health conditions) may allow the server to provide a notification on health-related events of the patient wearing the sensor for which the data came. For example, upon determining an arrhythmia condition from data received from a sensor, an external server may provide a notification indicating a cardiac event with respect to the wearer of the sensor that transmitted the data.

In some embodiments, the sensor may also include a temperature sensor, conductance sensor, a pressure sensor, a respiration sensor, SPO2, and/or a light sensor. For example, a respiration sensor can include an accelerometer configured to monitor the patient's chest movements, e.g., during certain portions of the day and/or night or during an RF measurement. For instance, a 3D multi-axis, multi-channel accelerometer can be configured to, on a first channel, monitor for a patient movement and/or posture, and on a second, different channel, monitor the chest movements of the patient to determine respiration rate and other related data. Alternatively, a respiration accelerometer can be provided in the device that is separate from a posture sensing accelerometer. In some examples, the respiration rate measurement can be based on the operation of a tri-axis micro-electromechanical system (MEMS) accelerometer within the device mounted on the patient's torso. The accelerometer can measure projections of the gravity vector on its intrinsic axes. From these measurements, a respiration rate can be derived based on measured quasi-periodic changes of the projections that occur due to respiration movements of the patient's rib cage.

In other examples, the respiration rate and/or other respiration data can be derived from the RF signals themselves. For example, dedicated respiration circuitry can be provided and/or the processor can be configured with instructions to cause the processor to monitor the reflected RF waves as described herein and determine respiration rate and related data therefrom. In some embodiments, respiration characteristics such as exhale vs. inhale times can also be measured via an accelerometer, and health conditions such as sleep apnea may be detected from accelerometer measurements.

FIGS. 5-10 illustrate various methods and processes that utilize RF signals for placement or verification of placement of an RF-based cardiac device at a prescribed anatomical location.

In some embodiments, an RF-based cardiac device can include a sensor and patch analogous to that described above and be configured to perform one or more aspects of the methods or processes illustrated in FIGS. 5-10. For example, the RF-based cardiac device can include an RF sensor that is reversibly mechanically coupled to a patch. As discussed above, an RF-based cardiac device can be configured to determine a thoracic and/or pulmonary fluid level and aid in the management of heart failure. The RF-based cardiac device may include a patch and sensor that includes an RF-sensor.

In examples, the patch is configured to have dimensions of about 3 cm by 10 cm. In an example implementation, the patch is configured to have dimensions of about 10 cm by 10 cm. In another example implementation, the patch is configured to have dimensions of about 12 cm by 12 cm. In some examples, the patch is configured to have dimensions of about 15 cm by 15 cm. In some examples, the patch is configured to have dimensions of about 20 cm by 20 cm.

In some example implementations, the patch is configured to have dimensions of about 6 cm by 12 cm. In an example, the patch is configured to have dimensions of about 8 cm by 15 cm. In another example, the patch is configured to have dimensions of about 10 cm by 20 cm. The above dimensions are provided as examples. Other dimensions are possible and within scope of this disclosure.

In examples, the RF sensor that includes the RF radar transmitting antenna and/or receiving antenna can be sized to measure around 2×2×2 cm. In examples, the RF sensor that includes the RF radar transmitting antenna and/or receiving antenna can be sized to measure around 5×5×5 cm. In examples, the RF sensor that includes the RF radar transmitting antenna and/or receiving antenna can be sized to measure around 10×10×10 cm. In examples, the RF sensor that includes the RF radar transmitting antenna and/or receiving antenna can be sized to measure around 5 cm of length x around 10 cm of width, and around 2 cm of height. In examples, the RF sensor that includes the RF radar transmitting antenna and/or receiving antenna can be sized to measure around 10 cm of length x around 5 cm of width, and around 5 cm of height. In examples, the RF sensor that includes the RF radar transmitting antenna and/or receiving antenna can be sized so as to have a length of between 1-20 cm, a width of between 1-20 cm, and a height of between 1-20 cm. In examples, the RF-based cardiac device can include ECG electrodes to detect electrical signals from a patient's heart. Data collected by the RF radar and associated circuitry can be analyzed as described herein to determine a measure of thoracic fluid content in a predetermined portion within the patient's chest (e.g., within the lung).

For example, the RF transmitting and RF receiving antenna can be two separate antennas on the RF sensor facing towards the patient's skin. For example, each of the RF transmitting and RF receiving antenna can include roughly 0.5×2 cm antennas. In some examples, each of the RF transmitting and RF receiving antenna can include roughly 1×3 cm antennas. In some examples, each of the RF transmitting and RF receiving antenna can include roughly 2×4 cm antennas. In some examples, a single RF antenna can be used, and such RF antenna can be switched between transmitting and receiving, based on the RF module in accordance with the operation described herein. As described herein, the antennas can be located on an underside of the device, facing the patient's skin, enabling them to be as close to the patient's body as possible. In some embodiments, one or more aspects of the methods or processes illustrated in FIGS. 5-10 can be performed by a processor in electrical communication with the RF-based cardiac device.

In some embodiments, the patch can be configured to be adhesively coupled to the skin of the patient. For example, the patch can be positioned at or about the prescribed anatomical location such that the RF sensor that engages with the patch is placed at the prescribed anatomical location.

As discussed above, in some embodiments, the RF sensor can be configured to be mechanically coupled to the patch. The RF sensor can include at least one antenna and an associated transmitter circuitry and/or receiver circuitry. Transmitter circuitry can be configured to transmit RF waves into a patient's body and receiver circuitry can be configured to receive the reflected RF waves from the patient's body. In some embodiments, a single antenna can include transmitter circuitry and receiver circuitry. In some embodiments, a single antenna can include one of transmitter circuitry and receiver circuitry. In some embodiments, the RF sensor can include one antenna or a plurality of antennas.

At least one processor can be in electrical communication with the RF sensor. The processor can be located in the sensor, gateway, or a server in communication with the RF sensor. The processor can be configured to generate one or more RF metric signals.

For example, the processor can be configured to analyze one or more properties of the transmitted RF signal and the received reflected RF signal in order to generate images and generate RF-based metrics for the tissue.

Generating the RF-based metrics can involve processing of the transmitted RF signal and/or the received reflected RF signal. For example, processing may involve applying band pass filtering, fast-Fourier transform, and the like to the transmitted and/or received RF signals. The transmitted and/or received RF signals may include characteristics of the RF signal including a phase and an amplitude. Changes in the phase and/or amplitude of the RF signal between the transmitted and/or received RF signals can be used to generate RF-based metrics. A series of RF-based metrics or generated metrics for a span of time can form an RF metric signal. RF-based metrics can include thoracic fluid measures, lung fluid parameters, and/or cardiac output.

FIG. 5A illustrates a method of obtaining RF-based metrics using an RF-based physiological monitoring device. The method includes directing the transmission of RF waveforms towards a target area in the patient's body 501 via a transmitter or the like. For example, the target area can include the patient's thoracic cavity, internal tissue, and the like. The method can also include the step of receiving reflected RF waveforms at a receiver 503. The method can include the step of generating one or more RF-based metrics based on the transmitted RF waveforms and the received reflected RF waveforms 505. For example, the phase and/or amplitude of the received reflected RF waveforms can be compared with that of the transmitted RF waveforms to generate one or more RF-based metrics. The RF-based metrics can include thoracic fluid measures, lung fluid parameters, and/or cardiac output.

RF-based metrics can be used to determine one or more thoracic fluid parameters of the patient. For example, thoracic fluid parameters can include one or more metrics indicative of a computed or estimated fluid content of tissue in the patient's body, such as the estimated fluid content in a patient's lungs. The determined one or more thoracic fluid parameters can include a thoracic fluid index (TFI). The RF-based physiological monitoring device can be configured to perform one or more RF-based measurements of a thoracic fluid level of the patient over a given time period (e.g., 1 minute, 10 minutes, 1 hour, 24 hours, etc., including values and subranges therebetween) to determine a thoracic fluid level. In some embodiments, the thoracic fluid level is an average of RF-based measurements taken over the given time period.

As noted above, FIG. 5A illustrates a method performed by an RF-based physiological monitoring device placement verification system. As illustrated in FIG. 5A, a processor can be in electrical communication with radio frequency (RF) antenna and associated transmitter/receiver circuitry in the RF-based physiological monitoring device. The at least one processor can be configured to perform the method of FIG. 5A. In some embodiments, the at least one processor may include one or more processors locally located on the RF-based physiological monitoring device. In some embodiments, the at least one processor may include one or more processors locally located on the RF-based monitoring device (e.g., to perform steps 501 and 503, discussed above) and one or more processors located remotely from the RF sensor (e.g., to perform step 505 discussed above). As discussed above, the RF-based physiological monitoring device can include at least one antenna which is disposed within the attachment structure and configured to direct radio frequency (RF) waves in a range from 500 MHz to 5 GHz towards a subject or patient proximate the at least one antenna and receive the reflected RF waves from the subject. The device can include device circuitry including at least one controller that is electrically coupled to the at least one antenna. The device circuitry can be configured to control the generation and transmission of the RF waves 501 and receipt of reflected RF waves 503. The device circuitry can further process reflected RF waves into RF metric signals corresponding to the reflected RF waves. The system further can also include communications circuitry configured for wired or wireless transmission of the RF metric signals to an external entity, such as a remote processing server. Thus, the generation of one or more RF-based metrics using the RF metric signals (e.g., produced using the transmitted RF signals and the received RF signals, as described) 505 may occur at the device circuitry and/or at the remote processing server.

As described above, in some implementations, an RF-based physiological monitoring device can be configured to be worn over long periods of time (e.g., days, weeks, months) for substantially continuous wear. A user or patient can be required to replace the RF-based physiological monitoring device or components of the device during the continuous wear. For example, the user may be required to exchange the patch component and/or sensor component of the device. The user may then be required to re-attach the RF-based physiological monitoring device after removing the RF-based physiological monitoring device to perform actions (e.g., showering, replacing one or more components of the device). To illustrate, the patch component may include an adhesive that will eventually start to peel from the patient's skin and/or need to be changed to avoid skin irritation. Thus, the user may need to remove the patch component, place a new patch component on their skin, and reattach the sensor component to the replacement patch component. Changes in the positioning of the RF-based physiological monitoring device with respect to the prescribed anatomical location or the location that the RF-based physiological device was previously attached to can lead to inaccurate readings or hinder the determination of trends in the physiological parameters determined by the device. Accordingly, it may be advantageous for an RF-based physiological monitoring device to be re-placed to the prescribed anatomical location and/or to the original placement of the monitoring device on the patient's skin.

Examples of the one or more RF-based metrics generated based on the transmitted RF waveforms and the received reflected RF waveforms of the method illustrated in FIG. 5A are further illustrated in FIG. 5B. These one or more RF-based metrics may be used to determine whether the patient replaced the physiological monitoring device at the same location, as discussed in further detail below.

In some examples, RF-based metrics associated with an internal tissue can include changes that are determined via analysis of transmitted and/or reflected RF waves as a radar cross section (RCS) of a patient's artery within a target tissue region (e.g., within a thoracic cavity of the patient). In examples, RCS includes a measure of the amount of waves reflected back to an RF wave source in relation to the transmitted waves, and includes information about the reflecting artery or arteries.

For example, the RCS includes a measure of an amount of the RF wave scattered by the artery as a function of the observation angle. In this respect, RCS can be expressed as a limit of {4*pi*d²(Sr/Si)} as d tends to infinity in the far field. Here, d is the distance of the artery from the antennas, and Sr and Si are reflected and incident power densities (W/m²) of the RF waves, respectively. The RCS is given in units of m² and may be interpreted as a cross-sectional area of a perfectly reflecting sphere and which would isotopically re-radiate the incident field. The artery, in this regard, can be approximated as a circular cylinder. The RF receiver, in some examples, receives only a portion of the reflected waves from the artery. The time-varying RCS measure can be calculated by modeling the artery as a circular cylindrical model of infinite length relative to the wavelength of the RF signals. For example, during a cardiac cycle, since the diameter of the artery varies over time, the time-varying RCS of the artery extracted from an analysis of the waves reflected back to the RF sensor may change over time as well, providing information on whatever caused the change in artery diameter. In each patient, the RCS information can be determined in a first position as a baseline reference. When the patch is re-placed, the updated RCS information associated with the replaced patch can be compared to the baseline reference, in accordance with the process described below (See, e.g., steps 603 to 609 of FIG. 6 below).

When a patch is re-placed in the same position, the re-placed patch is expected to exhibit the same or substantially the same RCS information or signal. The pattern of the received RF signal for the replaced patch is expected to substantially overlap with a baseline signal or with the pattern for the prior patch. RF metrics including RCS information or signal for a replaced patch can be compared with the RF metrics and RCS information determined just prior to patch replacement. A significant change in the RF metrics or RCE information may be indicative of improper patch placement, such as the patch being improperly located or that the replacement patch is not properly attached.

FIG. 5B illustrates RF metrics for an initial patch 502, a first replacement patch 504, and a second replacement patch 506. As shown in FIG. 5B, the average RF patterns (corresponding to the RCS information) can be plotted across the frequency domain. For example, RF metric signals produced from the transmitted RF signals and the received RF signals as described above may be in the form of RF signal amplitude over time. A processor (e.g., at the sensor, at a gateway device, and/or at a remote server) may apply a fast-Fourier transform to the RF metric signals to produce, for example, the graph shown in FIG. 5B. This transformation may be repeated, for instance, for RF metric signals acquired for an initial patch placement (502), RF metric signals acquired for a first replacement patch placement (504), and RF metric signals acquired for a second replacement patch placement (506). In implementations, using the frequency domain may require less processing power than using the time domain.

As an illustration, the sensor may acquire the initial patch signals 502 when the patient places the initial patch and/or when the patient indicates to the sensor that the patient is going to imminently change the patch. For instance, the patient may indicate to the sensor that the patient is going to imminently change the patch by pressing a button on the sensor (e.g., button 350 shown in FIG. 3B), by interacting with the gateway device 130 (e.g., by selecting a screen or a button on the gateway device, based on user interfaces displayed to the patient, for instance, as part of an application executing on the gateway device 130), and/or the like. In another example, the sensor may identify the last RF metric signals produced before the patient changed the patch. To illustrate, the sensor may determine that the patient has changed the patch based on a loss of contact between the sensor and the patient (e.g., based on a threshold of noise in ECG signals detected by the sensor, based on a threshold of noise in the RF metric signals, and/or the like). The sensor may then identify the last RF measurement that took place before the patient changed the patch.

The sensor may also acquire the first replacement patch (504) and/or the second replacement patch (506) signals based on determining that the patient has reattached the sensor to the patient's body, for instance, based on the reacquisition of ECG signals, RF signals, and/or the like below the threshold of noise. In examples, the sensor may determine the reacquisition of ECG signals and immediately initiate RF measurements to produce the first replacement patch and second replacement patch signals 504 and 506, respectively. Alternatively, in examples, the first replacement patch and/or second replacement patch signals 504 and 506, respectively, may be the first scheduled RF measurements taken after the patch has been changed. For instance, a processor (e.g., at the sensor, the gateway device, and/or the remote server) may determine that the patch has been changed based on a loss and reestablishment of contact between the sensor and the patient (e.g., based on noise in the ECG signal increasing above a threshold and then below the threshold, based on noise in the RF signal increasing above a threshold and then below the threshold, and/or the like). The processor may then identify the RF measurements taken the most recently after the patch change. In the illustrated example, the first replacement patch 504 has a pattern substantially similar to the pattern displayed by the initial patch signal 502, while the second replacement patch signal 506 has a distinctly different pattern (e.g., offset by phase, as shown).

In some implementations the RF metrics, including RCS information, can be compared to determine whether there is a significant change in the RCS information signal. In examples, a significant change can be quantified by computing the distance between the RCS pattern for the replacement patch and the RCS pattern of the initial patch and determining whether it exceeds a threshold.

A method for calculating the change in the RCS pattern for the replacement patch and the RCS pattern of the initial patch can include calculating the Euclidean distance and in vectors spaces the L² distance therebetween. The calculated Euclidean distance can be compared across a threshold to determine whether the replacement patch is positioned at substantially the same location as the original patch. Additional details regarding example thresholds are described in further detail below.

In some implementations, the Euclidean distance can be expressed as the square root of the summation indexed over the frequency range of the square of the difference between the RF value for the replacement patch value at the index and the RF value for the initial patch value at the index. In this way, the calculation can be represented as the following, where i represents the summation index, Oᵢ represents the original pattern (e.g., the initial patch signal 502), and Nᵢ represents the new pattern (e.g., the first replacement patch signal 504 or the second replacement patch signal 506):

$$Threshold < \sqrt{{\sum }_{i = 1}^{64} {( {N}_{i} - {O}_{i} )}^{2}}$$

As indicated above, the summation index is across, for example, bins 0 to 63 that represent a predetermined frequency range. For example, the frequency range may be from about (e.g., within a predetermined amount of variance or error, such as 1%, 2%, 3%, 4%, 5%, 10%, and/or the like) 0 GHz to 3.0 GHz. As another example, the frequency range may be from about 0.25 GHz to 2.75 Hz. As another example, the frequency range may be from about 0.5 GHz to 2.50 GHz. As another example, the frequency range may be from about 0.5 GHz to 2.25 GHz. As another example, the frequency range may be from about 0.5 GHz to 2.10 GHz. As another example, the frequency range may be from about 0.5 GHz to 2.00 GHz. In implementations, the summation index may be across more or fewer bins.

In one or more implementations, the predetermined threshold value is dynamically calculated by the at least one processor based on a statistical analysis of the signal environment. During an initial calibration period, or during the first period of time, the system is configured to capture a plurality of RF-based metrics (e.g., n=10 to 100 measurement cycles) to establish a baseline of signal variance at the prescribed anatomical location. The at least one processor, which may include hardware-based logic, an FPGA, an ASIC, or a microprocessor, calculates a standard deviation (σ) of the Euclidean distances between these baseline frequency patterns.

The predetermined threshold value may be defined as a function of the standard deviation to account for physiological noise or patient postural shifts. In implementations, the threshold is set to a value within a range of 1.5σ to 3.0σ, and in certain cases, the threshold is set to twice (2σ) the calculated standard deviation. For instance, if the baseline standard deviation of the RF-based metric is measured at a value V, the threshold for a “successful” re-placement may be set at 2V. If the distance between the post-replacement RF-based metric and the pre-replacement RF-based metric (e.g., the re-placement score) is less than or equal to 2V, the placement can be deemed acceptable.

Alternative implementations and numerical ranges include the following.

Weighted averages: As an alternative to a standard deviation, the processor may use a weighted moving average of the RF-based metric signals, where more recent signals prior to patch removal are weighted more heavily (e.g., a weight of 0.6 to 0.8) than older signals.

Coefficient of variation: The threshold may also be expressed as a percentage of the mean signal intensity. In such cases, the threshold transgresses if the re-placement score deviates by more than 5% to 15% from the mean of the first one or more RF-based metrics.

Frequency bin sensitivity: While a summation index may be across bins 0 to 63, the processor may be configured to prioritize specific frequency bins (e.g., those within the 1.0 GHz to 2.0 GHz range) that are more sensitive to thoracic fluid changes, applying a higher multiplier to the standard deviation in those specific bins.

The process for determining the re-placement score and threshold comparison may be implemented in combinational and sequential logic circuits within an ASIC to minimize power consumption and ensure real-time alert provision. Alternatively, the logic may be implemented as a firmware-level comparative algorithm that triggers an interrupt-driven alert if the re-placement score fails to transgress the threshold (e.g., remains within a predetermined acceptable statistical range of the original placement).

The method illustrated in FIG. 6 can be performed to verify re-placement of the RF-based physiological monitoring device to a prescribed anatomical location and/or to the original placement of the monitoring device on the patient's skin.

In a first step 601, first one or more RF metric signals can be generated. The one or more RF metric signals can be generated during a first period of time based on the transmitted and/or reflected RF waves associated with a prescribed anatomical location of the patch on the body of the patient 601. In some aspects, the first one or more RF metrics can be configured for determining first one or more thoracic fluid parameters of the patient. In examples, the first time that the one or more RF metric signals are generated can correspond to when an RF-based physiological monitoring device is attached on a patient's skin. In examples, the first time that the one or more RF metric signals are generated can correspond to the last measurement readings that the RF-based physiological monitoring device took before being removed from the patient's skin (e.g., to replace an adhesive patch).

In a second step 603, the processor can be further configured to generate second one or more RF metric signals. The second one or more RF metric signals can be generated during a second period of time subsequent to the first period of time based on the transmitted and/or reflected RF waves associated with a re-placement of the patch at a proposed anatomical location of the body of the patient. In other words, the second one more RF metric signals can correspond to a re-placement or re-attachment of the patch. Because the re-attachment of the patch on the skin is performed by a human, and thus may be prone to error, in some uses the proposed anatomical location for re-attachment may not correspond exactly to the prescribed anatomical location or placement of the patch during the generation of the first one or more RF metric signals. Accordingly, the disclosed method can be used to verify the proposed anatomical location for re-attachment of the patch.

At both the first time and the second time, the corresponding one or more RF metrics signals can be determined based on the transmitted and/or reflected RF waves in accordance with the methods described above such as by comparing amplitudes and phases in the transmitted and reflected RF waves.

In some embodiments, the one or more RF metric signals are configured for determining one or more thoracic fluid parameters of the patient. The RF metric signals can include one or more values indicative of a cardiac output and/or a thoracic fluid value based on the transmitted and received RF waves. Cardiac output can be indicative of the amount of blood a heart pumps in one minute. A thoracic fluid value can be indicative of an amount of fluid in the pleural space or thoracic cavity. The thoracic fluid value can be indicative of one or both of intracellular and extracellular fluid volume.

The first period of time and the second period of time can each span any suitable amount of time. For example, each period of time can correspond to one second, two seconds, three seconds, minutes, hours, days, or the like. The transmitted RF waveforms and their corresponding reflected RF waveforms can be determined over the period of time.

In a third step 605, the processor can be further configured to analyze the first and second one or more RF metrics in order to determine a re-placement score associated with the re-placement of the patch at a proposed anatomical location of the body of the patient. The re-placement score can be indicative of how closely the position of the RF-based device at the proposed anatomical location corresponding to the second time matches the position of the RF-based device at the first location corresponding to the first time. The first location can correspond to the prescribed anatomical location and/or where the patch was previously attached to the skin.

In some embodiments, the re-placement score can include a numerical value in the range of 0 to 1, 0 to 10, 0 to 100, or the like, including values and subranges therebetween. The numerical value can be indicative of how close the proposed anatomical location is to the prescribed anatomical location. In some embodiments, the re-placement score can include a categorial value such as “good” or “bad” that indicates whether the RF-based device should be re-attached at the proposed anatomical location. In some embodiments, the re-placement score can include a percentage difference from the prescribed anatomical location and/or the prior placement location.

In some embodiments, the re-placement score can be based on an analysis of the one or more RF metric signals at the first time in comparison to the one or more RF metric signals at the second time. For example, the one or more RF metric signals can correspond to a thoracic fluid index, and the re-placement score can be determined based on a comparison of the thoracic fluid index being measured at the first time and the second time. A percentage can be calculated based on the difference between the thoracic fluid index at the second time and the thoracic fluid index at the first time scaled by the thoracic fluid index. Alternatively, a numerical value can be calculated based on the difference between the thoracic fluid index at the second time and the thoracic fluid index at the first time.

In some embodiments, the re-placement score can be based on an analysis of the reflected waveforms corresponding to the first time and the second time. For example, the re-placement score can be determined from a comparison of the amplitude and/or phase associated with the reflected RF waves corresponding to the generated first one or more RF metric signals with the amplitude and/or phase associated with the reflected RF waves corresponding to the generated second one or more RF metric signals. For example, in some implementations, the re-placement score can be based on a comparison between the average amplitude of the reflected RF waves corresponding to the first time and the second time. In some implementations, the re-placement score can be based on a comparison between the average phase of the reflected RF waves corresponding to the first time and the second time. The average can be taken for a suitable time period. The suitable time period may span 5 minutes, 10 minutes, 30 minutes, 1 hour, 2 hours, 3 hours, . . . 10 hours, 12 hours, . . . 24 hours and/or the like, including values and subranges therebetween.

In some implementations, the re-placement score can be based on a comparison between the amplitude and/or phase of the reflected RF waves corresponding to the second time with a baseline amplitude and/or baseline phase associated with the reflected RF waves corresponding to the first time. The baseline amplitude and/or baseline phase associated with the reflected RF waves corresponding to the first time can be determined during a training or initiation period of the RF-based physiological device, be based on historical waveform values, and/or a population of waveform values derived from a plurality of patients. The baseline phase and/or amplitude values can be determined by the RF-based device for an initial time period. The initial time period may be 5 minutes, 10 minutes, 30 minutes, 1 hour, 2 hours, 3 hours, . . . 10 hours, 12 hours, . . . 24 hours and/or the like, including values and subranges therebetween.

In a fourth step 607, the processor can be configured to determine whether the re-placement score transgresses a predetermined threshold value. In some implementations, where the re-placement score is based on a phase and/or amplitude of the reflected RF waves corresponding to the second time, the predetermined threshold value can be indicative of when the phase and/or amplitude of the reflected RF wave associated with the second RF metric is within a predetermined percentage of the phase and/or amplitude of the reflected RF wave associated with the first RF metric. Similar percentages can be determined for the average phase and average amplitude, the baseline phase and baseline amplitude and the like. The predetermined percentage can be any suitable percentage, for example 10 percent or less, a threshold percentage of 9 percent, 8 percent, 7 percent, 6 percent, 5 percent, 4 percent, 3 percent, 2 percent, or 1 percent, and/or the like, including values and subranges therebetween.

In some implementations, where the re-placement score is based on an RF metric value, such as the thoracic fluid index, the predetermined threshold value can be applied to the re-placement score directly. For example, the absolute difference in the generated one or more RF metric signals during the first period of time can be compared to the generated one or more RF metric signals during the second period of time and the difference can be compared to the predetermined threshold. Alternatively, the predetermined threshold can indicate if the difference is more than a set percentage of the RF metric value.

In some implementations, the re-placement score can be determined based on the difference of the two values determined by the RF-based device. The determined re-placement score can be compared to a predetermined threshold value. Alternatively, the predetermined threshold can correspond to a percentage value indicating an acceptable tolerance for the location. For example, the predetermined threshold can be a percentage of the range of the RF-metric and correspond to a set percentage variation from the last measured RF-metric. For example, the percentage variation for the variation tolerance can be any suitable percentage, for example, 10 percent or less, a threshold percentage of 9 percent, 8 percent, 7 percent, 6 percent, 5 percent, 4 percent, 3 percent, 2 percent, or 1 percent, and/or the like, including values and subranges therebetween.

In a fifth step, the processor can be configured to provide an alert 609 indicating that the re-placement of the patch at the proposed anatomical location is unacceptable based on determining that the re-placement score transgresses the predetermined threshold value. An alert indicting that the re-placement of the patch at the proposed anatomical location is unacceptable can include an audible sound, a visual indicator, and/or a vibration. As an illustration, the monitoring device 110 or the transmission device 130 may emit an audible alert (e.g., a chime, a statement indicating that the patient needs to change the patch location, and/or the like). As another illustration, the monitoring device 110 may include an indicator light that turns red. As another illustration, the monitoring device 110 may include a haptic motor that provides a vibration alert. As another illustration, the transmission device 130 may display a screen indicating that the patient needs to re-place the patch. The alert can be provided to a user device remote from and in communication with the at least one processor. For example, the alert can be provided on a user device associated with the patient, such as the patient's phone. Alternatively, the alert can be conveyed using the LEDs or other components of the RF based medical device. In some embodiments, the alert can be transmitted prior to the RF-device being semi-permanently engaged with the proposed location. In some embodiments, the alert can provide guidance on how the patch should be repositioned to meet the threshold. In some embodiments the alert can be provided in a graphical user interface. For instance, the graphical user interface may be displayed on the transmission device 130.

FIG. 7 illustrates a process or method for an RF-based physiological monitoring device placement verification system. Similar to what is described with respect to FIG. 6, a process for an RF-based physiological monitoring device placement verification system may generate first one or more RF metric signals during a first period of time based on the transmitted and/or reflected RF waves associated with a prescribed anatomical location of the patch on the body of the patient 701, generate second one or more RF metric signals during a second period of time subsequent to the first period of time based on the transmitted and/or reflected RF waves associated with a re-placement of the patch at a proposed anatomical location of the body of the patient 703, analyze the first and second one or more RF metrics to determine a re-placement score associated with the re-placement of the patch at a proposed anatomical location of the body of the patient 705, determine whether the re-placement score transgresses a predetermined threshold value 707, and provide an alert indicating that the re-placement of the patch at the proposed anatomical location is unacceptable based on determining that the re-placement score transgresses the predetermined threshold value 709. In some implementations, the one or more RF metrics can be configured for determining thoracic fluid parameters of the patient.

In some implementations, the re-placement score, and determining the re-placement score in relation to the predetermined threshold can be determined in accordance with the methods described with respect to FIG. 6.

Similar to the discussion of step 609, the alert provided in step 709 can indicate that the re-placement of the patch at the proposed anatomical location is unacceptable and can include an audible sound, a visual indicator, and/or a vibration. The alert can be provided to a user device remote from and in communication with the at least one processor. Alternatively, the alert can be conveyed using the LEDs or other components of the RF based medical device. In some embodiments, the alert can be transmitted prior to the RF-device being semi-permanently engaged with the proposed location. In some embodiments, the alert can provide guidance on how the patch should be repositioned to meet the threshold (e.g., a notification that the patch should be moved in a particular direction (e.g., “move patch towards armpit”), or by an amount of space (e.g., 3 cm).

FIG. 8 illustrates a process or method for an RF-based physiological monitoring device with a correction feature.

Similar to what is described with respect to FIGS. 6 and 7, a process for an RF-based physiological monitoring device with a correction feature may generate first one or more RF metric signals during a first period of time based on the transmitted and/or reflected RF waves associated with a prescribed anatomical location of the patch on the body of the patient, wherein the first one or more RF metrics is configured for determining first one or more thoracic fluid parameters of the patient 801, generate second one or more RF metric signals during a second period of time subsequent to the first period of time based on the transmitted and/or reflected RF waves associated with a re-placement of the patch at a proposed anatomical location of the body of the patient 803, analyze the first and second one or more RF metrics to determine a re-placement score associated with the re-placement of the patch at a proposed anatomical location of the body of the patient 805, determine whether the re-placement score transgresses a predetermined threshold value 807, and determine a correction responsive to determining the re-placement score transgresses a predetermined threshold value 809.

In some implementations, the re-placement score, and determining the re-placement score in relation to the predetermined threshold can be determined in accordance with the methods described with respect to FIGS. 6 and 7.

In some implementations, the processor can be used to determine a correction responsive to determining that the re-placement score transgresses the predetermined threshold value. The determined correction can then be applied to future RF metric signals. By applying a correction, the RF-based physiological monitoring device can provide improved accuracy and trending even when an RF-based physiological monitoring device is re-attached or re-placed to an improper location, a location that does not correspond to the prescribed anatomical location within a set tolerance amount.

In some implementations, the correction can be applied to future RF metrics. For example, one or more RF metrics generated during a third period of time subsequent to the second period of time can be modified by applying the determined correction to the RF metric generated by the reflected RF waves. In some implementations, the RF metric signals can correspond to a thoracic fluid value.

In some implementations, the processor can be configured to initiate a recalibration process for the RF-based physiological monitoring device when it is determined that the correction exceeds a recalibration threshold. The recalibration threshold can indicate when applying a correction value may not be suitable to provide accurate values for the RF metrics, and instead it would be more appropriate to perform a recalibration. In this manner, the system can determine whether the drift in the RF-based physiological monitoring device's positioning requires a recalibration or a correction. In some implementations, the recalibration threshold can be any suitable value. For example, in some implementations the recalibration threshold can be 10 percent or less of a maximum thoracic fluid value, a recalibration threshold percentage of 9 percent, 8 percent, 7 percent, 6 percent, 5 percent, 4 percent, 3 percent, 2 percent, or 1 percent, and/or the like, including values and subranges therebetween.

In some implementations, applying a correction to an RF metric can include determining an RF metric based on a corresponding reflected RF waveform, determining the correction value, and modifying the determined RF metric based on the determined correction value. In some embodiments, the determined correction value may correspond to a difference between RF metrics measured at two different times, a scaling, and/or the like. Applying the correction value may include modifying the determined RF metric by adding, subtracting, multiplying, and/or scaling the determined RF metric by the determined correction value.

The recalibration process for the RF-based physiological monitoring device may include determining locations of one or more components of the at least one radio frequency (RF) antenna and associated transmitter/receiver circuitry configured to transmit RF waves into and receive reflected RF waves from a body of the patient with respect to anatomical features of the patient.

FIGS. 9A and 9B illustrate a process for an RF-based physiological monitoring device which incorporates a placement verification system configured to provide an alert that a re-placement of the patch at a proposed anatomical location is acceptable or unacceptable, apply a correction for RF-metrics determined by a re-placed device, and/or send a signal to recalibrate the re-placed RF-based physiological monitoring device, in accordance with the processes and methods described herein. As shown in FIG. 9A a device may provide a patient with an alert to change a sensor and/or a patch of an RF-based physiological monitoring device 901, provide a patient a recommendation to record a first RF metric signal 903, receive a first RF metric signal 905, provide a patient with instructions for placing a new patch and/or sensor 907, provide a patient a recommendation to record a second RF metric signal prior to attaching the new patch and/or sensor. 909, receive a second RF metric signal 911, determine a re-placement score 913, provide an alert that re-placement of the patch at the proposed anatomical location is unacceptable based on the re-placement score 915, or alternatively, provide an alert that re-placement of the patch at the proposed anatomical location is acceptable if the re-placement score does not transgress the predetermined threshold value 917.

In some embodiments, the patient or user may attach the new patch and/or sensor prior to checking for a re-placement score. Accordingly, after a patient is provided with instructions for placing a new patch and/or sensor 907, the device may receive confirmation that the new patch and/or sensor is attached 919. Subsequently, the device may provide a patient instructions to record a second RF metric signal 921, receive the second RF metric signal 923, determine a correction 925, and apply a correction to one or more future RF metric signals of the RF-based physiological monitoring device if the correction is below a recalibration threshold 927, or alternatively, recalibrate the device if the correction is below a recalibration threshold 929.

Additionally, in some embodiments, the device may determine a correction 925 after determining that the re-placement of the patch at the proposed anatomical location is acceptable 917.

For example, an RF-based device can be configured to determine a thoracic fluid metric in the form of a thoracic fluid index (TFI) (e.g., TFI range between 0.5 and 1.5). A first TFI value (e.g., 1) can be received prior to a patient replacing an existing patch and/or sensor with a new patch and/or sensor. A second TFI value (e.g., 1.25) can be received at a second time at a proposed anatomical location, prior to the new patch and/or sensor being attached. A re-placement score based on a difference can be determined (e.g., 1.25−1=0.25). Whether the re-placement score transgresses a predetermined threshold (e.g., 10 percent variation for the TFI range) can be determined. In the present example, the re-placement score (e.g., 0.25) transgresses a predetermined threshold (e.g., 10 percent variation for the range), and an alert that re-placement of the patch at the proposed anatomical location is unacceptable can be provided.

FIG. 10 illustrates graphical user interfaces (GUIs) that can be used for an RF-based physiological monitoring device which incorporates a placement verification system. For example, as illustrated in FIG. 10, a device may include a GUI that prompts a user to change their sensor and/or patch 1001. Similarly, the GUI may prompt the user to take an RF metric signal reading. Alternatively, the device displaying the GUI may prompt the RF-based physiological monitoring device to take an RF metric signal reading. For example, the transmission device 130 may display the GUI and may immediately before or simultaneously with the GUI prompt the monitoring device 110 to take an RF metric signal reading. The GUI can also display one or more videos, prompts, and the like that provide the patient with instructions on how to place a new patch and/or sensor 1003. To illustrate, the GUI may display a video showing the patient how to replace an adhesive patch, a series of prompts guiding the patient through the patch replacement process, and/or the like. After a patient's new patch is placed and confirmed, the GUI can also provide additional graphical notifications to the patient 1005. For example, the GUI may indicate to the patient when the patch re-attachment is acceptable and when the patch re-attachment is unacceptable and needs to be moved.

The GUI can include one or more displays that show RF signals and other measured physiological parameters (e.g., movement of the heart, heart rate, ECG, cardiovascular parameters, respiratory parameters, and/or the like). The GUI may also provide information regarding the antenna positions, circuitry, and/or the like. The GUI may also include one or more interactive buttons to provide patient feedback, confirmation and/or selection of items displayed on the GUI.

FIG. 11 is a scatter plot illustrating experimental data of RF-based metrics, e.g., thoracic fluid content (TFC) values, over time. The experimental data demonstrates example sensitivity of RF-based metrics to patch placement and the detection of placement “jumps” or gaps in the values as a result of patch removals and/or replacements. In accordance with the placement verification system described herein, as shown in FIG. 11, the processor of the fluid monitoring system generates a plurality of RF-based metrics, illustrated here as individual scatter plot dots 1100 representing TFC values. These RF-based TFC values are derived from RF waves transmitted into and reflected from the patient's body (e.g., the thoracic region).

The horizontal axis 1104 represents time (e.g., in this illustration, shown in days), while the vertical axis 1108 represents a calculated TFC value. The vertical lines 1120 (e.g., lines 1120a, 1120b, 1120c, 1120d, and 1120e) indicate patch attachment tests/events. When a patient removes the sensor/patch and re-attaches it (e.g., without replacing the patch), or performs a replacement of the patch at a proposed anatomical location, the processor generates RF-based metrics as shown and, as such, corresponding TFC values for the new anatomical location.

During a first period of time 1112, the patch is at a first prescribed anatomical location, generating a baseline of first RF-based metrics as first TFC values. In this regard, the system calculates a first mean 1116 of such first RF-based metrics, here the first TFC values (e.g., as indicated by the upper horizontal line), representing a first stable physiological state at the first prescribed anatomical location. During a second period of time 1113, the patch is at a second prescribed anatomical location as indicated by the vertical lines 1120b and 1120c (e.g., two closely run attachment tests). Following the placement at the second prescribed anatomical location, the system generates a number of second RF-based metrics as second TFC values. In this regard, the system calculates a second mean 1128 of such second RF-based metrics, here the second TFC values (e.g., as indicated by the lower horizontal line 1128), representing a second stable physiological state at the second prescribed anatomical location.

As seen between Day 4 and Day 5, a significant “jump” or gap is visibly discernable, as illustrated by label 1132. In this regard, a processor of the fluid monitoring system can analyze such first and second RF-based metrics to determine a re-placement score. In one implementation, a score can be calculated based on the statistical deviation between the baseline measurements and the new measurements (the second RF-based metrics). For example, a “significant jump” can be flagged when a gap 1132 between the second mean 1128 (lower horizontal line) and the first mean 1116 is larger than a predetermined threshold.

In implementations, a predetermined threshold value can be based on the standard deviation of the measurements. As illustrated, a jump may be flagged if the difference between the means is larger than twice (2×) the standard deviation of the measurements prior to the test. Other thresholds may include a phase and/or amplitude variation of 10% or less, a numerical TFC value shift of 1.0 to 5.0 units, and/or a percentage shift in the RF metric signal ranging from 5% to 25%. If the re-placement score transgresses the threshold (e.g., the 2× standard deviation as noted above), the system can identify the placement as unacceptable. Conversely, if the score does not transgress the threshold, the system can provide an alert indicating the location is acceptable. For example, the sensor can light up a green LED to indicate acceptable placement or light up a red LED to indicate unacceptable placement. As another example, the sensor can issue an auditory confirmation to indicate acceptable placement (e.g., by issuing a confirmation of “patch placement acceptable”) or an auditory instruction to indicate unacceptable placement. In the event of a transgression, for instance, the system may determine a correction responsive to the score, such as instructing the patient to move the patch in a specific direction (e.g., “move 1 cm to the left” or “move 1 cm to the right” or “move 1 cm upwards” or “move 1 cm downwards”).

While FIG. 11 illustrates TFC values, the RF-based metrics may comprise various RF-based parameters, including RF-based magnitudes and phases of reflection coefficients, RF-based transmissions coefficients in multi-antenna implementations, and/or time-of-flight variations of the reflected RF envelope.

Advantages of the implementations herein include examples where a mechanical coupling between the sensor and the patch can be reversible, e.g., allowing the patient to remove the sensor, replace the adhesive patch, and re-attach the sensor. In this manner, the verification algorithm ensures that even if the adhesive is replaced, the RF “view” into the thoracic cavity remains consistent, ensuring longitudinal data integrity for chronic heart failure monitoring.

In other example scenarios, the system is not limited to patch replacements. For example, the system can be used to detect accidental patch peeling or shifting. The reporting of a “jump” as described above can be transmitted to an Independent Diagnostic Testing Facility (IDTF) or a clinician portal to trigger a follow-up with the patient.

While an RF-based physiological monitoring system is described herein, it is envisioned that a treatment system could incorporate an RF-based physiological monitoring system.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be an example and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure. Embodiments disclosed herein may also be combined with one or more features, as well as complete systems, devices, and/or methods, to yield yet other embodiments and inventions. Moreover, some embodiments may be distinguishable from the prior art by specifically lacking one and/or another feature disclosed in the particular prior art reference(s); i.e., claims to some embodiments may be distinguishable from the prior art by including one or more negative limitations.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1-41. (canceled)

42. An RF-based physiological monitoring device placement verification system, comprising:
a patch configured to be adhesively coupled to skin of a patient;
an RF sensor configured to be mechanically coupled to the patch, the RF sensor comprising
at least one radio frequency (RF) antenna and associated transmitter/receiver circuitry configured to transmit RF waves into and receive reflected RF waves from a body of the patient; and
at least one processor in electrical communication with the at least one radio frequency (RF) antenna and associated transmitter/receiver circuitry, the at least one processor configured to
generate first one or more RF metric signals during a first period of time based on at least one of the transmitted or reflected RF waves associated with a prescribed anatomical location of the patch on the body of the patient, wherein the first one or more RF metric signals is configured for determining first one or more thoracic fluid parameters of the patient;
generate second one or more RF metric signals during a second period of time subsequent to the first period of time based on at least one of the transmitted or reflected RF waves associated with a re-placement of the patch at a proposed anatomical location of the body of the patient,
analyze the first and second one or more RF metric signals to determine a re-placement score associated with the re-placement of the patch at the proposed anatomical location of the body of the patient;
determine whether the re-placement score transgresses a predetermined threshold value; and
provide an alert indicating whether the re-placement of the patch at the proposed anatomical location is acceptable based on whether the placement score does not transgress the predetermined threshold value.

43. The system of claim 42, wherein the mechanical coupling of the RF sensor to the patch is reversible.

44. The system of claim 42, wherein the patch is configured to maintain attachment to the skin of the patient in a range of about 4 days to about 10 days.

45. The system of claim 42, wherein the patch is configured to maintain attachment to the skin of the patient in a range of about 5 days to about 7 days.

46. The system of claim 42, wherein the at least one processor is further configured to determine at least one of a cardiac output or a thoracic fluid value based on the transmitted and received RF waves.

47. The system of claim 42, wherein the at least one processor is remote from the RF sensor.

48. The system of claim 42, wherein the at least one radio frequency (RF) antenna and associated transmitter/receiver circuitry comprises at least one transmitter and at least one receiver antenna.

49. The system of claim 42, wherein the at least one radio frequency (RF) antenna and associated transmitter/receiver circuitry comprises at least one antenna configured to transmit the RF waves and receive the reflected RF waves.

50. The system of claim 42, wherein the alert is provided to a user device remote from and in communication with the at least one processor.

51. The system of claim 50, wherein the user device comprises the patient's phone.

52. The system of claim 42, wherein the alert comprises at least one of an audible sound, a visual indicator, or a vibration.

53. The system of claim 42, wherein the alert indicates that the re-placement of the patch at the proposed anatomical location is acceptable based on the placement score not transgressing the predetermined threshold value.

54. The system of claim 42, wherein the alert indicates the re-placement of the patch at the proposed anatomical location is not acceptable based on the placement score transgressing the predetermined threshold value.

55. The system of claim 54, wherein the alert provides guidance on repositioning the patch.

56. The system of claim 42, wherein determining a re-placement score comprises comparing at least one of a phase or an amplitude of one or more reflected RF waveforms associated with the first and second one or more RF metrics.

57. The system of claim 42, wherein determining a re-placement score comprises determining at least one of an average phase or an average amplitude of reflected RF waveforms associated with the first and second one or more RF metrics.

58. The system of claim 42, wherein determining a re-placement score comprises comparing the at least one of a phase or an amplitude of reflected RF waveforms associated with the first and second one or more RF metrics to at least one of a baseline phase or a baseline amplitude, respectively.

59. The system of claim 58, wherein the at least one of the baseline phase or the baseline amplitude is determined based on one or more prior historical reflected RF waveforms for the patient.

60. The system of claim 58, wherein the at least one of the baseline phase or the baseline amplitude is determined based one or more prior historical reflected RF waveforms for one or more populations of patients.

61. The system of claim 42, wherein the predetermined threshold value is indicative of a phase and/or amplitude variation of 10 percent or less.
